(19) United States
(12) Patent Application Publication
(10) Pub. No.: US 2012/0157451 A1
(43) Pub. Date: Jun. 21, 2012

(54) RAF INHIBITOR COMPOUNDS AND METHODS OF USE THEREOF

(75) Inventors: Stefan Gradl, South San Francisco, CA (US); Joachim Rudolph, South San Francisco, CA (US); Li Ren, Boulder, CO (US)

(73) Assignee: GENENTECH, INC, South San Francisco, CA (US)

(21) Appl. No.: 13/393,069

(22) PCT Filed: Aug. 27, 2010
(86) PCT No.: PCT/US10/46975

§ 371 Date: Feb. 28, 2012

Related U.S. Application Data
(60) Provisional application No. 61/238,103, filed on Aug. 28, 2009.

(51) Int. Cl. A61K 31/519; A61P 13/12; A61P 35/00; C07D 487/04; A61K 31/5377

(52) U.S. Cl. 514/233.2; 544/281; 514/259.3; 514/252.16; 544/117

(57) ABSTRACT

Compounds of Formula (I) are useful for inhibition of Raf kinases. Methods of using compounds of Formula (I) and stereoisomers, tautomers and pharmaceutically acceptable salts thereof, for in vitro, in situ, and in vivo diagnosis, prevention or treatment of such disorders in mammalian cells, or associated pathological conditions are disclosed.

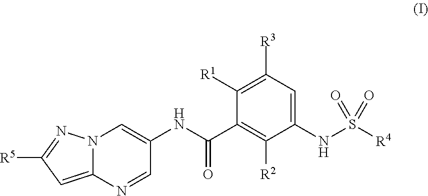

### PRIORITY OF INVENTION

This application claims priority under 35 U.S.C. 119(e) from U.S. Provisional Patent Application No. 61/238,103, filed 28 Aug. 2009, the content of which is incorporated herein in its entirety.

### BACKGROUND OF THE INVENTION

1. Field of the Invention

Compounds, pharmaceutical compositions comprising the compounds, a process for making the compounds and the use of the compounds in therapy are provided herein. More particularly, certain substituted pyrazolo[1,5-a]pyrimidine compounds useful for inhibiting Raf kinase and for treating disorders mediated thereby are disclosed herein.

2. Description of the State of the Art

The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability.

Small molecule inhibitors of B-Raf are being developed for anticancer therapy. Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example RAF-265, GSK-2118436, PLX-3603, PLX-4032 and XL-281. Other B-Raf inhibitors are also known, see for example, U.S. Patent Application Publication 2006/0189627, U.S. Patent Application Publication 2006/0281751, U.S. Patent Application Publication 2007/0049603, U.S. Patent Application Publication 2009/0176809, International Patent Application Publication WO 2007/002325, International Patent Application Publication WO 2007/002433, International Patent Application Publication WO 2008/028141, International Patent Application Publication WO 2008/079903, International Patent Application Publication WO 2008/079906 and International Patent Application Publication WO 2009/012283.

Kinase inhibitors are known, see for example, International Patent Application Publication WO 2006/066913, International Patent Application Publication WO 2008/028617 and International Patent Application Publication WO 2008/079909.

### SUMMARY OF THE INVENTION

Compounds that are inhibitors of Raf kinases, particularly B-Raf inhibitors, are described herein. Certain hyperproliferative disorders are characterized by the over activation of Raf kinase function, for example by mutations or over expression of the protein. Accordingly, the compounds are useful in the treatment of hyperproliferative disorders, such as cancer.

More specifically, one aspect provides compounds of Formula I:

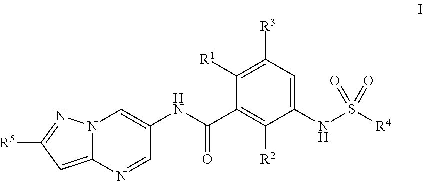

and stereoisomers, tautomers and pharmaceutically acceptable salts thereof, wherein R¹, R², R³, R⁴ and R⁵ are as defined herein.

Another aspect provides methods of preventing or treating a disease or disorder modulated by B-Raf, comprising administering to a mammal in need of such treatment an effective amount of a compound of Formula I, a stereoisomer, tautomer or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect provides methods of preventing or treating cancer, comprising administering to a mammal in need of such treatment an effective amount of a compound of Formula I, a stereoisomer, tautomer or pharmaceutically acceptable salt thereof, alone or in combination with one or more additional compounds having anti-cancer properties.

Another aspect provides a method of treating a hyperproliferative disease in a mammal comprising administering a therapeutically effective amount of a compound of Formula I, a stereoisomer, tautomer or pharmaceutically acceptable salt thereof to the mammal.

Another aspect provides methods of preventing or treating kidney disease, comprising administering to a mammal in need of such treatment an effective amount of a compound of Formula I, a stereoisomer, tautomer or pharmaceutically acceptable salt thereof, alone or in combination with one or more additional compounds. Another aspect provides methods of preventing or treating polycystic kidney disease, comprising administering to a mammal in need of such treatment an effective amount of a compound of Formula I, a stereoisomer or pharmaceutically acceptable salt thereof, alone or in combination with one or more additional compounds.

Another aspect provides the use of a compound of Formula I, a stereoisomer, tautomer or pharmaceutically acceptable salt thereof in the manufacture of a medicament for the treatment of a hyperproliferative disease.

Another aspect provides the compounds of Formula I, a stereoisomer, tautomer or pharmaceutically acceptable salt thereof for use in therapy.

Another aspect provides the compounds of Formula I, a stereoisomer, tautomer or pharmaceutically acceptable salt thereof for use in the treatment of a hyperproliferative disease. In a further embodiment, the hyperproliferative disease may be cancer (or still further, a specific cancer as defined herein).

Another aspect provides the compounds of Formula I, a stereoisomer, tautomer or pharmaceutically acceptable salt thereof for use in the treatment of a kidney disease. In a further embodiment, the kidney disease may be polycystic kidney disease.

Another aspect provides the use of a compound of Formula I, a stereoisomer, tautomer or pharmaceutically acceptable salt thereof in the manufacture of a medicament for the treatment of a hyperproliferative disease. In a further embodiment, the hyperproliferative disease may be cancer (or still further, a specific cancer as defined herein).

Another aspect provides the use of a compound of Formula I, a stereoisomer, tautomer or pharmaceutically acceptable salt thereof in the manufacture of a medicament for the treatment of a kidney disease. In a further embodiment, the kidney disease may be polycystic kidney disease.

Another aspect provides the use of a compound of Formula I, a stereoisomer, tautomer or pharmaceutically acceptable salt thereof in the manufacture of a medicament, for use as a B-Raf inhibitor in the treatment of a patient undergoing cancer therapy.

Another aspect provides the use of a compound of Formula I, a stereoisomer, tautomer or pharmaceutically acceptable salt thereof in the manufacture of a medicament, for use as a B-Raf inhibitor in the treatment of a patient undergoing polycystic kidney disease therapy.

Another aspect provides a pharmaceutical composition comprising a compound of Formula I, a stereoisomer, tautomer or pharmaceutically acceptable salt thereof for use in the treatment of a hyperproliferative disease.

Another aspect provides a pharmaceutical composition comprising a compound of Formula I, a stereoisomer, tautomer or pharmaceutically acceptable salt thereof for use in the treatment of cancer.

Another aspect provides a pharmaceutical composition comprising a compound of Formula I, a stereoisomer, tautomer or pharmaceutically acceptable salt thereof for use in the treatment of polycystic kidney disease.

Another aspect provides a pharmaceutical composition comprising a compound of Formula I, a stereoisomer, tautomer or pharmaceutically acceptable salt thereof and a pharmaceutically acceptable carrier or excipient.

Another aspect provides intermediates for preparing compounds of Formula I. Certain compounds of Formula I may be used as intermediates for other compounds of Formula I.

Another aspect includes processes for preparing, methods of separation, and methods of purification of the compounds described herein.

### DETAILED DESCRIPTION OF THE INVENTION

Reference will now be made in detail to certain embodiments, examples of which are illustrated in the accompanying structures and formulas. While enumerated embodiments will be described, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### Definitions

The term “alkyl” includes linear or branched-chain radicals of carbon atoms. In one example, the alkyl radical may be one to six carbon atoms (C₁-C₆). In other examples, the alkyl radical may be C₁-C₅, C₁-C₄ or C₁-C₃. Some alkyl moieties have been abbreviated, for example, methyl (“Me”), ethyl (“Et”), propyl (“Pr”) and butyl (“Bu”), and further abbreviations are used to designate specific isomers of compounds, for example, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”) and the like. The abbreviations are sometimes used in conjunction with elemental abbreviations and chemical structures, for example, methanol (“MeOH”) or ethanol (“EtOH”).

Additional abbreviations used throughout the application may include, for example, benzyl (“Bn”), phenyl (“Ph”) and acetate (“Ac”).

The term “alkenyl” includes linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical may be two to six carbon atoms (C₂-C₆). In other examples, the alkenyl radical may be C₂-C₅, C₂-C₄ or C₂-C₃.

The term “alkynyl” includes linear or branched-chain monovalent hydrocarbon radical with at least one site of unsaturation, i.e., a carbon-carbon triple bond, wherein the alkynyl radical may be optionally substituted independently with one or more substituents described herein. In one example, the alkynyl radical may be two to six carbon atoms (C₂-C₆). In other examples, the alkynyl radical may be C₂-C₅, C₂-C₄ or C₂-C₃.

The term “alkoxy” refers to a radical of the formula —O-(alkyl), wherein the alkyl may be substituted.

The term “cycloalkyl” refers to a non-aromatic, saturated or partially unsaturated hydrocarbon ring group, wherein the cycloalkyl group may be optionally substituted independently with one or more substituents described herein. In one example, the cycloalkyl group may be 3 to 6 carbon atoms (C₃-C₆). In other examples, cycloalkyl may be C₅-C₆, C₃-C₄ or C₃-C₅.

The terms “heterocycle” and “heterocyclic” include saturated or a partially unsaturated four to seven membered rings containing one, two or three heteroatoms selected from the group consisting of oxygen, nitrogen and sulfur, with the remaining atoms being carbon. In one example, the heterocyclic may be a 3 to 6 membered ring. In other examples, the heterocyclic may be a 4 to 6 membered ring or a 5 to 6 membered ring.

The term “heteroaryl” includes five to six membered aromatic rings containing one, two or three heteroatoms selected from the group consisting of oxygen, nitrogen and sulfur, with the remaining atoms being carbon. In one example, the heteroaryl may be a 5 to 6 membered ring.

The term “halogen” refers to F, Cl, Br or I.

The terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound of Formula I that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, skin cancer, including melanoma, as well as head and neck cancer.

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound described herein.

The compounds described herein also include other salts of such compounds that are not necessarily pharmaceutically acceptable salts, and which may be useful as intermediates for preparing and/or purifying compounds described herein and/or for separating enantiomers of compounds described herein.

The term “mammal” means a warm-blooded animal that has or is at risk of developing a disease described herein and includes, but is not limited to, guinea pigs, dogs, cats, rats, mice, hamsters, and primates, including humans.

B-Raf Inhibitor Compounds

Provided herein are compounds, and pharmaceutical formulations thereof, that are potentially useful in the treatment of diseases, conditions and/or disorders modulated by B-Raf.

One embodiment provides compounds of Formula I:

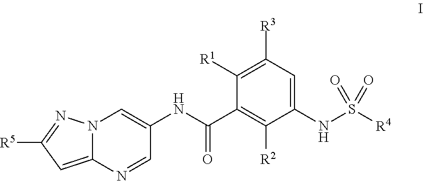

and stereoisomers, tautomers and pharmaceutically acceptable salts thereof, wherein:

R¹ and R² are independently selected from hydrogen, halogen, C₁-C₃ alkyl and C₁-C₃ alkoxy;

R³ is selected from hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl, or NR^{a}R^{b}, wherein the cycloalkyl, alkyl, alkenyl, alkynyl, phenyl and heteroaryl are optionally substituted with OR^{c}, halogen, phenyl, C₃-C₄ cycloalkyl, or C₁-C₄ alkyl optionally substituted with halogen;

R⁵ is selected from hydrogen, C₁-C₆ alkyl, OR^{d}, NR^{e}R^{f}, SR⁹, C₃-C₆ cycloalkyl, phenyl, a 4-6 membered heterocyclic and a 5-6 membered heteroaryl, wherein the alkyl, cycloalkyl and heterocyclic are optionally substituted with one to three R^{h} groups, and the phenyl and heteroaryl are optionally substituted with one to three Rⁱ groups;

R^{a} and R^{b} are independently selected from hydrogen and C₁-C₅ alkyl optionally substituted with halogen, or

R^{a} and R^{b} together with the nitrogen to which they are attached form a 4 to 6 membered heterocyclic ring;

R^{c} is hydrogen, phenyl and C₁-C₄ alkyl optionally substituted with oxo;

R^{d} is C₁-C₆ alkyl optionally substituted with OH or OCH₃;

R^{e} and R^{f} are independently selected from hydrogen and C₁-C₆ alkyl;

R^{g} is C₁-C₆ alkyl;

each R^{h} is independently selected from halogen, oxo, C₁-C₆ alkyl, C₁-C₆ alkoxy and a 4-6 membered heterocyclic, wherein the alkyl, alkoxy and heterocyclic are optionally substituted with R^{j};

each Rⁱ is independently selected from halogen, C₁-C₆ alkyl, C₁-C₆ alkoxy and a 4-6 membered heterocyclic, wherein the alkyl, alkoxy and heterocyclic are optionally substituted with R^{k};

R^{j} is selected from halogen, OH, oxo and C₁-C₃ alkyl; and

R^{k} is selected from halogen, OH and C₁-C₃ alkyl.

In certain embodiments, R¹, R² and R³ are independently selected from hydrogen, halogen and C₁-C₃ alkyl. In certain embodiments, R¹, R² and R³ are independently selected from hydrogen, halogen and methyl. In certain embodiments, R¹, R² and R³ are independently selected from hydrogen, F, Cl and methyl.

In certain embodiments, R¹ and R² are independently selected from halogen, and R³ is hydrogen. In certain embodiments; R¹ and R² are independently selected from F and Cl, and R³ is hydrogen.

In certain embodiments, R¹ and R² are independently selected from hydrogen, halogen, C₁-C₃ alkyl and C₁-C₃ alkoxy.

In certain embodiments, R¹ and R³ are independently selected from hydrogen, halogen or C₁-C₃ alkyl, and R² is Cl. In certain embodiments, R¹ and R³ are independently selected from hydrogen, F, Cl and methyl, and R³ is Cl.

In certain embodiments, R¹ is hydrogen, halogen, C₁-C₃ alkyl or C₁-C₃ alkoxy.

In certain embodiments, R¹ is hydrogen.

In certain embodiments, R¹ is halogen. In certain embodiments, R¹ is F or Cl.

In certain embodiments, R¹ is C₁-C₃ alkyl. In certain embodiments, R¹ is methyl.

In certain embodiments, R² is hydrogen, halogen, C₁-C₃ alkyl or C₁-C₃ alkoxy.

In certain embodiments, R² is hydrogen.

In certain embodiments, R² is halogen. In certain embodiments, R² is F or Cl.

In certain embodiments, R² is C₁-C₃ alkyl. In certain embodiments, R² is methyl.

In certain embodiments, R² is Cl.

In certain embodiments, R² is hydrogen.

In certain embodiments, R³ is hydrogen, halogen or C₁-C₃ alkyl.

In certain embodiments, R³ is hydrogen.

In certain embodiments, R³ is halogen. In certain embodiments, R³ is F or Cl.

In certain embodiments, R¹ and R² are F, and R³ is hydrogen.

In certain embodiments, R¹ is F; R² is Cl; and R³ is hydrogen.

In certain embodiments, R¹ is Cl; R² is F; and R³ is hydrogen.

In certain embodiments, R¹ is F, and R² and R³ are hydrogen.

In certain embodiments, R¹ and R³ are hydrogen, and R² is F.

In certain embodiments, R² and R³ are F, and R¹ is hydrogen.

In certain embodiments, R¹ is Cl, and R² and R³ are hydrogen.

In certain embodiments, R¹, R² and R³ are F.

In certain embodiments, R¹ is F; R² is methyl; and R³ is hydrogen.

In certain embodiments, R¹ is methyl; R² is F; and R³ is hydrogen.

In certain embodiments, R¹ is F, and R² and R³ are hydrogen.

In certain embodiments, R¹ is Cl, and R² and R³ are hydrogen.

In certain embodiments, R² is F, and R¹ and R³ are hydrogen.

In certain embodiments, the residue:

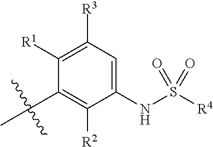

of Formula I, wherein the wavy line represents the point of attachment of the residue in Formula I, is selected from:

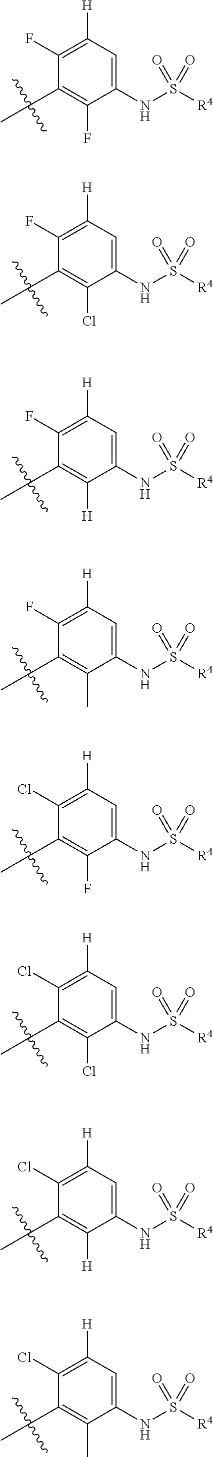

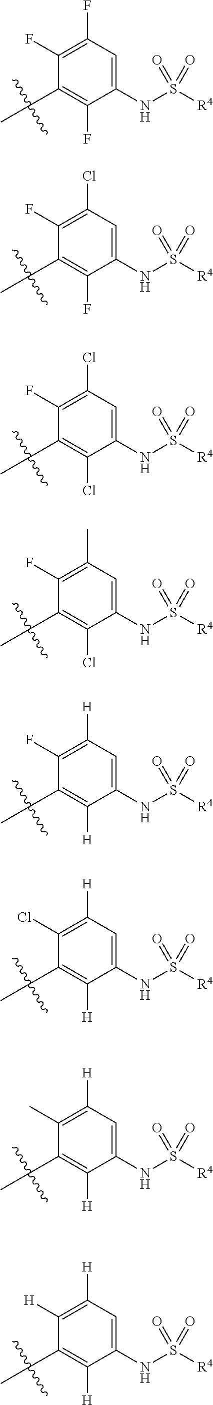

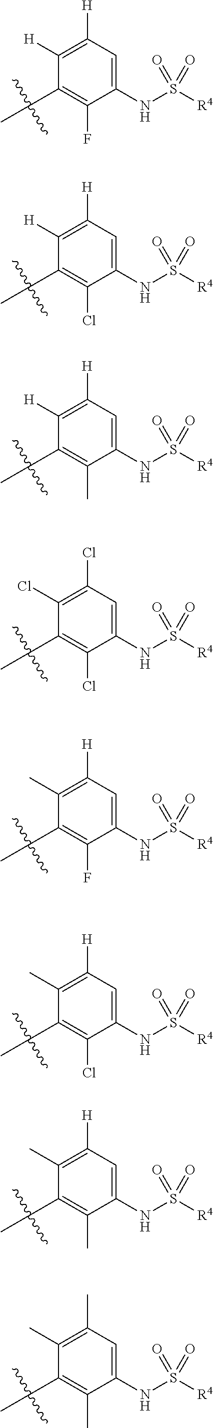

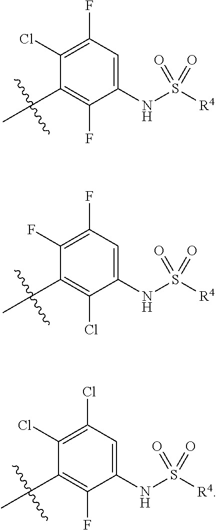

In certain embodiments, R⁴ is C₃-C₅ cycloalkyl, C₁-C₆ alkyl, C₂-C₆ alkenyl, C₂-C₆ alkynyl, phenyl, a 5-6 membered heteroaryl, or NR^{a}R^{b}, wherein the cycloalkyl, alkyl, alkenyl, alkynyl, phenyl and heteroaryl are optionally substituted with OR^{c}, halogen, phenyl, C₃-C₄ cycloalkyl, or C₁-C₄ alkyl optionally substituted with halogen.

In certain embodiments, R⁴ is selected from C₁-C₆ alkyl optionally substituted with halogen, and NR^{a}R^{b}. In certain embodiments, R⁴ is selected from propyl, isobutyl, —CH₂CH₂CH₂F, —N(CH₃)CH₂CH₃ and pyrrolidin-1-yl.

In certain embodiments, R⁴ is cyclopropyl, ethyl, propyl, butyl, isobutyl, —CH₂CH₂CH₂OH, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃, —N(CH₃)CH₂CH₃, —NHCH(CH₃)₂, —NHCH₂CHF₂, —N(CH₃)₂ or pyrrolidin-1-yl.

In certain embodiments, R⁵ is selected from hydrogen, C₁-C₆ alkyl, OR^{d}, NR^{e}R^{f}, SR^{g}, C₃-C₆ cycloalkyl, phenyl, a 4-6 membered heterocyclic and a 5-6 membered heteroaryl, wherein the alkyl, cycloalkyl and heterocyclic are optionally substituted with one to three R^{h} groups, and the phenyl and heteroaryl are optionally substituted with one to three Rⁱ groups.

In certain embodiments, R^{d} is C₁-C₆ alkyl optionally substituted with OH or OCH₃.

In certain embodiments, R^{e} and R^{f} are independently selected from hydrogen and C₁-C₆ alkyl.

In certain embodiments, R^{g} is C₁-C₆ alkyl.

In certain embodiments, each R^{h} is independently selected from halogen, oxo, C₁-C₆ alkyl, C₁-C₆ alkoxy and a 4-6 membered heterocyclic, wherein the alkyl, alkoxy and heterocyclic are optionally substituted with R^{j}. In certain embodiments, each R^{h} is independently selected from halogen, C₁-C₆ alkyl and a 4-6 membered heterocyclic, wherein the alkyl and heterocyclic are optionally substituted with R^{j}.

In certain embodiments, R^{j} is selected from halogen, OH, oxo and C₁-C₃ alkyl. In certain embodiments, R^{j} is selected from OH and C₁-C₃ alkyl.

In certain embodiments, each Rⁱ is independently selected from halogen, C₁-C₆ alkyl, C₁-C₆ alkoxy and a 4-6 membered heterocyclic, wherein the alkyl, alkoxy and heterocyclic are optionally substituted with R^{k}. In certain embodiments, each Rⁱ is independently selected from halogen, C₁-C₆ alkyl and a 4-6 membered heterocyclic, wherein the alkyl and heterocyclic are optionally substituted with R^{k}.

In certain embodiments, R^{k} is selected from halogen, OH and C₁-C₃ alkyl. In certain embodiments, R^{k} is selected from OH and C₁-C₃ alkyl.

In certain embodiments, R⁵ is selected from hydrogen, methyl, ethyl, CF₃, —OCH₃, —OCH₂CH₃, —OCH(CH₃)₂, —OCH₂CH₂OH, —OCH₂CH₂OCH₃, —NHCH₃, —NHCH(CH₃)₂, —SCH₃, cyclopropyl, cyclopentyl, phenyl, 4-chlorophenyl, 3-fluorophenyl, 4-fluorophenyl, 4-methylphenyl, 3-(4-methylpiperazin-1-yl)phenyl, tetrahydrofuran-3-yl, pyrrolidin-1-yl, morpholin-4-yl, piperidin-4-yl, 1-methyl-1H-pyrazol-4-yl, 1-(2-hydroxyethyl)-1H-pyrazol-4-yl and pyridin-3-yl.

In certain embodiments, R⁵ is hydrogen.

In certain embodiments, R⁵ is C₁-C₆ alkyl optionally substituted with one to three R^{h} groups. In certain embodiments, R⁵ is selected from methyl, ethyl and CF₃.

In certain embodiments, R⁵ is OR^{d}. In certain embodiments, R^{d} is C₁-C₆ alkyl optionally substituted with OH or OCH₃. In certain embodiments, R⁵ is selected from —OCH₃, —OCH₂CH₃, —OCH(CH₃)₂, —OCH₂CH₂OH and —OCH₂CH₂OCH₃.

In certain embodiments, R⁵ is NR^{e}R^{f}. In certain embodiments, R^{e} and R^{f} are independently selected from hydrogen and C₁-C₆ alkyl. In certain embodiments, R⁵ is selected from —NHCH₃ and —NHCH(CH₃)₂.

In certain embodiments, R⁵ is SR^{g}. In certain embodiments, R^{g} is C₁-C₆ alkyl. In certain embodiments, R⁵ is —SCH₃.

In certain embodiments, R⁵ is C₃-C₆ cycloalkyl optionally substituted with one to three R^{h} groups. In certain embodiments, R⁵ is C₃-C₆ cycloalkyl. In certain embodiments, R⁵ is cyclopropyl or cyclopentyl.

In certain embodiments, R⁵ is phenyl optionally substituted with one to three Rⁱ groups. In certain embodiments, each Rⁱ is independently selected from halogen, C₁-C₆ alkyl and a 4-6 membered heterocyclic, wherein the alkyl and heterocyclic are optionally substituted with R^{k}, and wherein the heterocyclic contains one, two or three heteroatoms selected from oxygen, nitrogen and sulfur. In certain embodiments, each Rⁱ is independently selected from halogen, C₁-C₆ alkyl and a 4-6 membered heterocyclic, wherein the alkyl and heterocyclic are optionally substituted with R^{k}, and wherein the heterocyclic is piperazinyl. In certain embodiments, R⁵ is selected from phenyl, 4-chlorophenyl, 3-fluorophenyl, 4-fluorophenyl, 4-methylphenyl and 3-(4-methylpiperazin-1-yl)phenyl.

In certain embodiments, R⁵ is a 4-6 membered heterocyclic optionally substituted with one to three R^{h} groups. In certain embodiments, R⁵ is a 4-6 membered heterocyclic, wherein the heterocyclic contains one, two or three heteroatoms selected from oxygen, nitrogen and sulfur. In certain embodiments, R⁵ is a 4-6 membered heterocyclic, wherein the heterocyclic is selected from tetrahydrofuranyl, pyrrolidinyl, morpholinyl and piperidinyl. In certain embodiments, R⁵ is tetrahydrofuran-3-yl, pyrrolidin-1-yl, morpholin-4-yl and piperidin-4-yl.

In certain embodiments, R⁵ is a 5-6 membered heteroaryl optionally substituted with one to three Rⁱ groups. In certain embodiments, R⁵ is a 5-6 membered heteroaryl optionally substituted with one to three Rⁱ groups, wherein the heteroaryl contains one, two or three heteroatoms selected from the group consisting of oxygen, nitrogen and sulfur. In certain embodiments, R⁵ is a 5-6 membered heteroaryl optionally substituted with one to three Rⁱ groups, wherein the heteroaryl is selected from pyrazolyl and pyridinyl. In certain embodiments, R⁵ is selected from 1-methyl-1H-pyrazol-4-yl, 1-(2-hydroxyethyl)-1H-pyrazol-4-yl and pyridin-3-yl.

It will be appreciated that certain compounds described herein may contain asymmetric or chiral centers, and therefore exist in different stereoisomeric forms. It is intended that all stereoisomeric forms of the compounds described herein, including but not limited to, diastereomers, enantiomers and atropisomers, as well as mixtures thereof such as racemic mixtures, form part of the present compounds.

In the structures shown herein, where the stereochemistry of any particular chiral atom is not specified, then all stereoisomers are contemplated and included as the compounds described herein. Where stereochemistry is specified by a solid wedge or dashed line representing a particular configuration, then that stereoisomer is so specified and defined.

It will also be appreciated that compounds of Formula I include tautomeric forms. Tautomers are compounds that are interconvertible by tautomerization. This commonly occurs due to the migration of a hydrogen atom or proton, accompanied by the switch of a single bond and adjacent double bond. Formation of tautomers of Formula I include, but not limited to, the sulfonamide position. The compounds of Formula I are intended to include all tautomeric forms.

It will also be appreciated that certain compounds of Formula I may be used as intermediates for further compounds of Formula I.

It will be further appreciated that the compounds described herein may exist in unsolvated as well as solvated forms with pharmaceutically acceptable solvents such as water, ethanol, and the like, and it is intended that the compounds embrace both solvated and unsolvated forms.

It will also be further appreciated that the compounds of Formula I include compounds that differ only in the presence of one or more isotopically enriched atoms. For example, compounds of Formulas I, wherein one or more hydrogen atoms are replaced deuterium or tritium, or one or more carbon atoms are replaced by a ¹³C- or ¹⁴C-enriched carbon are within the scope of this invention.

Synthesis of Compounds

Compounds described herein may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

For illustrative purposes, Schemes 1-6 show general methods for preparing the compounds described herein, as well as key intermediates. For a more detailed description of the individual reaction steps, see the Examples section below. Those skilled in the art will appreciate that other synthetic routes may be used to synthesize the compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

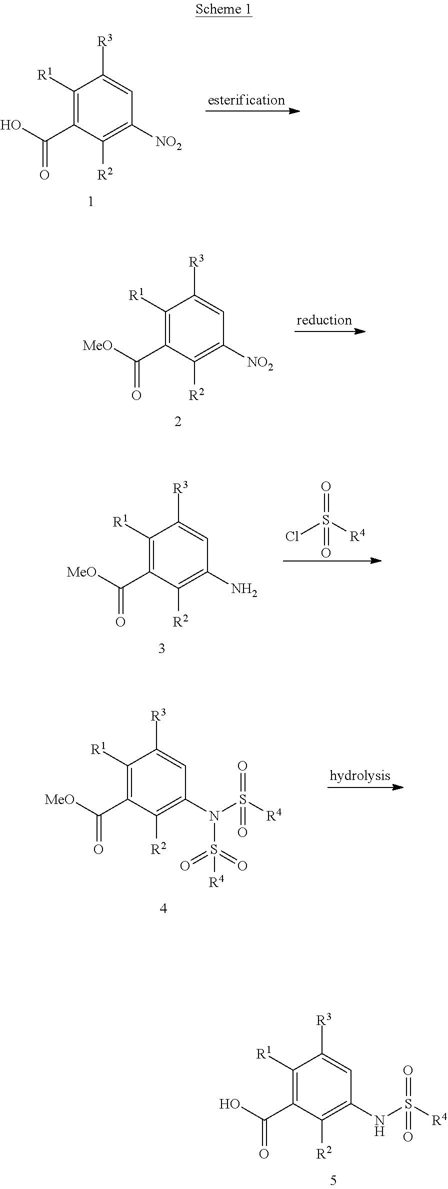

Scheme 1 shows a general method for preparing compound 5, wherein R¹, R², R³ and R⁴ are as defined herein. Benzoic acid 1 is esterified to methyl benzoate 2 by treatment with trimethylsilyl diazomethane in MeOH or via Fischer esterification conditions, such as treatment with trimethylsilyl chloride (“TMSCl”) in MeOH. Reduction of 2 is performed using a standard condition, such as treatment with Pd/C and H₂. Bis-sulfonamide 4 is obtained by treatment of aniline 3 with a sulfonyl chloride in the presence of a base, such as NEt₃, in an organic solvent, such as dichloromethane (“DCM”). Hydrolysis of 4 is accomplished under basic conditions, such as aqueous NaOH, in the appropriate solvent system, such as tetrahydrofuran (“THF”) and/or MeOH, to provide compound 5.

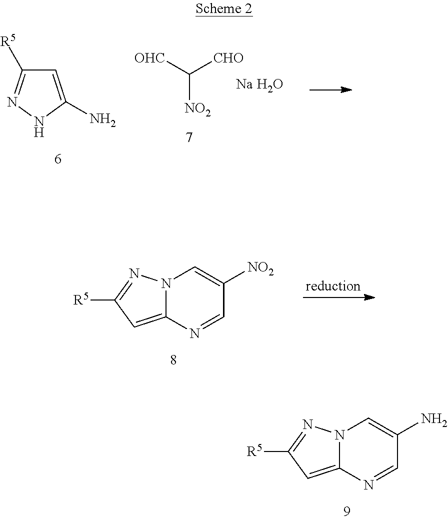

Scheme 2 shows a general method for preparing compounds 8, wherein R⁵ is as defined herein. Treatment of 3-substituted-1H-pyrazol-5-amine 6 with sodium nitromalonaldehyde monohydrate 7 in a suitable solvent, such as AcOH, at 25° C. affords 2-substituted-6-nitropyrazolo[1,5-a]pyrimidine 8. Standard reduction of the nitro functionality in compound 8, such as by treatment with Pd/C and H₂, affords 2-substituted-pyrazolo[1,5-a]pyrimidin-6-amine 9.

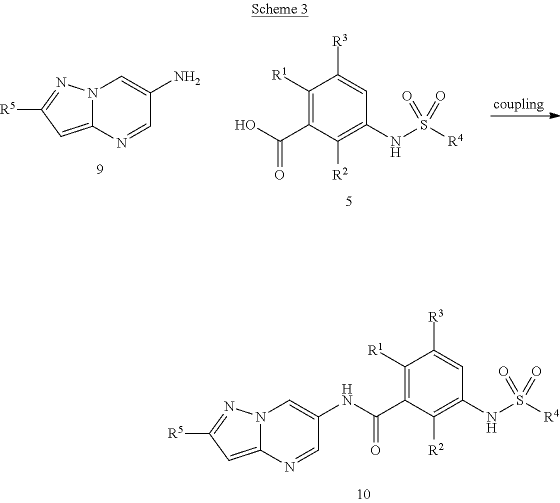

Scheme 3 shows a general method for preparing compound 10, wherein R¹, R², R³, R⁴ and R⁵ are as defined herein. Coupling of 2-substituted-pyrazolo[1,5-a]pyrimidin-6-amine 9 with acid 5 is performed with an activating reagent, such as N-(3-dimethylaminopropyl)-N′-ethylcarbodiimide hydrochloride (“EDCl”), in the presence of an additive, such as hydroxybenztriazole (“HOBt”), in a suitable solvent, such as dimethylformamide (“DMF”).

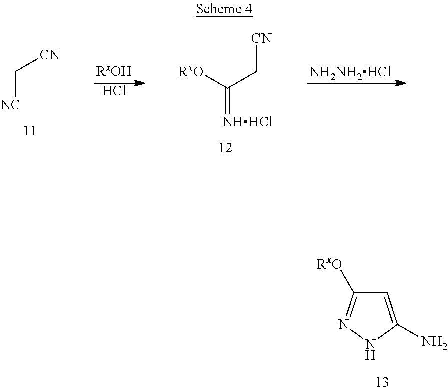

Scheme 4 shows a general method for preparing compound 13, wherein R^{x} is methyl or ethyl. Malononitrile 11 is converted to imino ester HCl salt 12 by treatment with alcohol R^{X}OH in the presence of HCl in an organic solvent, such as diethyl ether. Compound 12 is then condensed with hydrazine monohydrochloride in a suitable solvent, such as MeOH, to provide 3-alkoxyl-1H-pyrazol-5-amine 13.

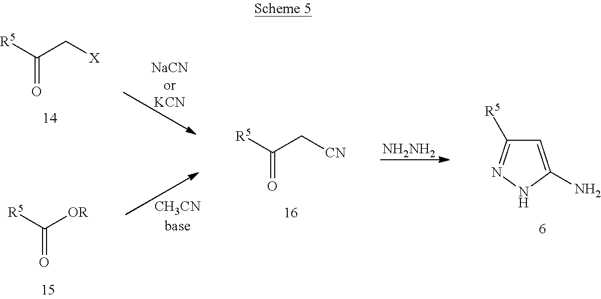

Scheme 5 shows a general method for preparing compound 6, wherein R⁵ is as defined herein. α-Cyanoketone 16 is prepared by reaction of an α-substituted ketone 14 with NaCN or KCN, wherein X is a halogen or a suitable leaving group, such as mesylate or tosylate, in a suitable organic solvent, such as DMF. Alternatively, α-cyanoketone 16 is prepared by treatment of ester 15 with CH₃CN and a suitable base, such as NaH or NaOt-Bu. Subjection of α-cyanoketone 16 to hydrazine in a solvent, such as EtOH, at 80° C. provides 3-substituted-1H-pyrazol-5-amine 6.

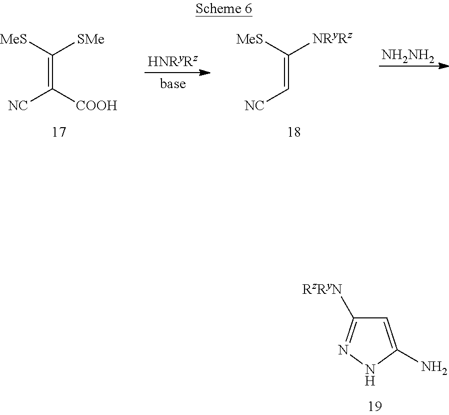

Scheme 6 shows a general method for preparing compound 19, wherein R^{y} is R^{e} and R^{z} is R^{f}, or R^{y} and R^{z} together with the nitrogen atom to which they are attached form a 4-6 membered heterocyclic optionally substituted with one to three R^{h} groups, such that the heterocyclic is attached via the nitrogen atom. Molononitrile 17 is converted to 3-amino-3-methylthio-acrylonitrile 18 by treatment with amine HNR^{y}R^{z} in the presence of a base, such as triethylamine, in an organic solvent, such as MeOH. Compound 18 is then condensed with hydrazine in a suitable solvent, such as EtOH, to provide 3-amino-1H-pyrazol-5-amine 19.

In preparing compounds of Formula I, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butyloxycarbonyl (“Boc”), benzyloxycarbonyl (“CBz”) and 9-fluorenylmethyleneoxycarbonyl (“Fmoc”). The need for such protection is readily determined by one skilled in the art. For a general description of protecting groups and their use, see T. W. Greene, et al. Greene's Protective Groups in Organic Synthesis. New York: Wiley Interscience, 2006.

Accordingly, another embodiment provides a process for preparing compounds of Formula I, comprising:

(a) coupling a compound of Formula 9:

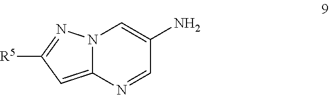

wherein R⁵ is as defined herein;

with a compound of Formula 5:

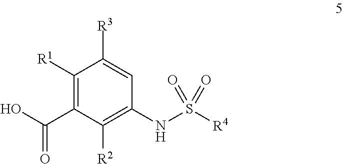

wherein R¹, R², R³ and R⁴ are as defined herein;

to provide a compound of Formula I.

In a further embodiment, the coupling is performed with an activating reagent. In a further embodiment, the activating reagent is EDCl.

In a further embodiment, the coupling is performed with an activating reagent in the presence of an additive. In a further embodiment, the activating reagent is EDC1. In a further embodiment, the additive is HOBt.

In a further embodiment, the coupling is performed with an activating reagent in the presence of an additive in a solvent. In a further embodiment, the activating reagent is EDCl. In a further embodiment, the additive is HOBt. In a further embodiment, the solvent is DMF.

Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds described herein may be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E. and Wilen, S. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., Ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Biological Evaluation

B-Raf mutant protein 447-717 (V600E) was co-expressed with the chaperone protein Cdc37, complexed with Hsp90 (Roe, S. Mark, et al. “The Mechanism of Hsp90 Regulation by the Protein Kinase-Specific Cochaperone p50^{cdc37}.” Cell. Vol. 116 (2004): pp. 87-98; Stancato, L F, et al. “Raf exists in a native heterocomplex with Hsp90 and p50 that can be reconstituted in a cell free system.” J. Biol. Chem. 268(29) (1993): pp. 21711-21716).

Determining the activity of Raf in the sample is possible by a number of direct and indirect detection methods (US 2004/0082014). Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. The activity/inhibition of V600E full-length B-Raf was estimated by measuring the incorporation of radio labeled phosphate from [γ-³³P]ATP into FSBA-modified wild-type MEK (see Biological Example 1).

Administration and Pharmaceutical Formulations

The compounds described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

A typical formulation is prepared by mixing a compound described herein and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound described herein or pharmaceutical composition thereof) or aid in the manufacturing of the pharmaceutical product (i.e., medicament).

One embodiment includes a pharmaceutical composition comprising a compound of Formula I, or a stereoisomer, tautomer or pharmaceutically acceptable salt thereof. A further embodiment provides a pharmaceutical composition comprising a compound of Formula I, or a stereoisomer, tautomer or pharmaceutically acceptable salt thereof, together with a pharmaceutically acceptable carrier or excipient.

Methods of Treatment with Compounds of the Invention

Also provided are methods of treating or preventing disease or condition by administering one or more compounds described herein, or a stereoisomer or pharmaceutically acceptable salt thereof. In one embodiment, a human patient is treated with a compound of Formula I, or a stereoisomer, tautomer or pharmaceutically acceptable salt thereof, and a pharmaceutically acceptable carrier, adjuvant, or vehicle in an amount to detectably inhibit B-Raf activity.

In another embodiment, a method of treating a hyperproliferative disease in a mammal comprising administering a therapeutically effective amount of the compound of Formula I, or a stereoisomer, tautomer or pharmaceutically acceptable salt thereof, to the mammal is provided.

In another embodiment, a method of treating cancer in a mammal comprising administering a therapeutically effective amount of the compound of Formula I, or a stereoisomer, tautomer or pharmaceutically acceptable salt thereof, to the mammal is provided.

In another embodiment, a method of treating a kidney disease in a mammal comprising administering a therapeutically effective amount of the compound of Formula I, or a stereoisomer, tautomer or pharmaceutically acceptable salt thereof, to the mammal is provided. In a further embodiment, the kidney disease is polycystic kidney disease.

In another embodiment, a method of treating or preventing cancer in a mammal in need of such treatment, wherein the method comprises administering to said mammal a therapeutically effective amount of a compound of Formula I, or a stereoisomer, tautomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment provides the use of a compound of Formula I, or a stereoisomer, tautomer or pharmaceutically acceptable salt thereof, in the manufacture of a medicament for the treatment of cancer.

In another embodiment, a method of treating or preventing kidney disease in a mammal in need of such treatment, wherein the method comprises administering to said mammal a therapeutically effective amount of a compound of Formula I, or a stereoisomer, tautomer or pharmaceutically acceptable salt thereof. In a further embodiment, the kidney disease is polycystic kidney disease.

In another embodiment, a method of treating or preventing a disease or disorder modulated by B-Raf, comprising administering to a mammal in need of such treatment an effective amount of a compound of Formula I, or a stereoisomer, tautomer or pharmaceutically acceptable salt thereof. Examples of such diseases and disorders include, but are not limited to, hyperproliferative diseases (including cancer) and kidney disease (including polycytic kidney disease).

Another embodiment provides the use of a compound of Formula I, or a stereoisomer, tautomer or pharmaceutically acceptable salt thereof, in the manufacture of a medicament for the treatment of a hyperproliferative disease.

Another embodiment provides the use of a compound of Formula I, or a stereoisomer, tautomer or pharmaceutically acceptable salt thereof, in the manufacture of a medicament for the treatment of cancer.

Another embodiment provides the use of a compound of Formula I, or a stereoisomer, tautomer or pharmaceutically acceptable salt thereof, in the manufacture of a medicament for the treatment of kidney disease. In a further embodiment, the kidney disease is polycystic kidney disease.

In another embodiment, a method of preventing or treating cancer, comprising administering to a mammal in need of such treatment an effective amount of a compound of Formula I, or a stereoisomer, tautomer or pharmaceutically acceptable salt thereof, alone or in combination with one or more additional compounds having anti-cancer properties.

Another embodiment of the present invention provides the compounds of Formula I for use in therapy.

Another embodiment of the present invention provides the compounds of Formula I for use in the treatment of a hyperproliferative disease. In a further embodiment, the hyperproliferative disease is cancer.

Another embodiment of the present invention provides the compounds of Formula I for use in the treatment of kidney disease. In a further embodiment, the kidney disease is polycystic kidney disease.

In one further embodiment, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In one further embodiment, the cancer is a sarcoma.

In another further embodiment, the cancer is a carcinoma. In one further embodiment, the carcinoma is squamous cell carcinoma. In another further embodiment, the carcinoma is an adenoma or adenocarcinoma.

Combination Therapy

The compounds described herein and stereoisomers and pharmaceutically acceptable salts thereof may be employed alone or in combination with other therapeutic agents for treatment. The compounds described herein may be used in combination with one or more additional drugs, for example an anti-hyperproliferative (or anti-cancer) agent that works through action on a different target protein. The second compound of the pharmaceutical combination formulation or dosing regimen preferably has complementary activities to the compound described herein, such that they do not adversely affect each other. Such molecules are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

A “chemotherapeutic agent” is a chemical compound useful in the treatment of cancer, regardless of mechanism of action. Chemotherapeutic agents include compounds used in “targeted therapy” and conventional chemotherapy. A number of suitable chemotherapeutic agents to be used as combination therapeutics are contemplated for use in the methods of the present invention. The present invention contemplates, but is not limited to, administration of numerous anticancer agents, such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene therapy reagents; antisense therapy reagents and nucleotides; tumor vaccines; inhibitors of angiogenesis, and the like.

Examples of chemotherapeutic agents include Erlotinib (TARCEVA®, Genentech/OSI Pharm.), Bortezomib (VELCADE®, Millennium Pharm.), Fulvestrant (FASLODEX®, Astra Zeneca), Sunitinib (SUTENT®, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (NEXAVAR®, Bayer), Irinotecan (CAMPTOSAR®, Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Raf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) PI3k/AKT/mTOR pathway inhibitors, including GDC-0941 (2-(1H-Indazol-4-yl)-6-(4-methanesulfonyl-piperazin-1-ylmethyl)-4-morpholin-4-yl-thieno[3,2-d]pyrimidine), XL-147, GSK690693 and temsirolimus; (xi) Ras/Raf/MEK/ERK pathway inhibitors; and (xii) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the Raf inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

### EXAMPLES

For illustrative purposes, the following Examples are included. However, it is to be understood that these Examples do not limit the invention and are only meant to suggest a method of practicing the invention. Persons skilled in the art will recognize that the chemical reactions described may be readily adapted to prepare a number of other compounds described herein, and alternative methods for preparing the compounds are deemed to be within the scope of this invention. For example, the synthesis of non-exemplified compounds may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds described herein.

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters) (unless otherwise stated). ¹H NMR spectra were recorded on a Varian instrument operating at 400 MHz. ¹H-NMR spectra were obtained as CDCl₃, CD₃OD, D₂O, (CD₃)₂SO, (CD₃)₂CO, C₆D₆, CD₃CN solutions (reported in ppm), using tetramethylsilane (0.00 ppm) or residual solvent (CDCl₃: 7.26 ppm; CD₃OD: 3.31 ppm; D₂O: 4.79 ppm; (CD₃)₂SO: 2.50 ppm; (CD₃)₂CO: 2.05 ppm; C₆D₆: 7.16 ppm; CD₃CN: 1.94 ppm) as the reference standard. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

### Biological Example 1

### B-Raf IC₅₀ Assay Protocol

Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. Catalytically active human recombinant B-Raf protein is obtained by purification from sf9 insect cells infected with a human B-Raf recombinant baculovirus expression vector.

The activity/inhibition of V600E full-length B-Raf was estimated by measuring the incorporation of radio labeled phosphate from [γ-³³P]ATP into FSBA-modified wild-type MEK. The 30-μL assay mixtures contained 25 mM Na Pipes, pH 7.2, 100 mM KCl, 10 mM MgCl₂, 5 mM β-glycerophosphate, 100 μM Na Vanadate, 4 μM ATP, 500 nCi [γ-³³P]ATP, 10/1 FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5× (20 μL of 30 nM and 1.5 μM, respectively) for 15 minutes, and the assay was initiated by the addition of 10 μL of 10 μM ATP. Following the 60-minute incubation, the assay mixtures were quenched by the addition of 100 μL of 25% TCA, the plate was mixed on a rotary shaker for 1 minute, and the product was captured on a Perkin-Elmer GF/B filter plate using a Tomtec Mach III Harvester. After sealing the bottom of the plate, 35 of Bio-Safe II (Research Products International) scintillation cocktail were added to each well and the plate was top-sealed and counted in a Topcount NXT (Packard).

The compounds of Examples 1-36 were tested in the above assay and found to have an IC₅₀ of less than 1 μM.

The compounds of Examples 1, 2, 3, 4, 5, 6, 7, 8, 9, 10, 11, 12, 13, 14, 15, 16, 18, 19, 20, 21, 22, 24, 25, 26, 27, 28, 29, 30, 31, 32, 33, 34, 35 and 36 were tested in the above assay and found to have an IC₅₀ of less than 300 nM.

The compounds of Examples 1, 2, 3, 5, 6, 7, 8, 9, 10, 11, 12, 13, 14, 15, 16, 19, 20, 21, 22, 25, 26, 28, 29, 30, 32, 33, 34 and 35 were tested in the above assay and found to have an IC₅₀ of less than 150 nM.

The following compounds were tested in the above assay. Some compounds were prepared multiple times and tested in the above assay multiple times. The below data is representative of those tests:

| Example # | IC₅₀ (nM) |
|---|---|
| Example 1 | 53.25 |
| Example 5 | 6.87 |
| Example 10 | 14.67 |
| Example 11 | 4.09 |

| Example # | IC₅₀ (nM) |
|---|---|
| Example 17 | 366.85 |
| Example 22 | 7.78 |
| Example 24 | 259.5 |
| Example 34 | 46.06 |

### Biological Example 2

### Cellular ERK 1/2 Phosphorylation Assay

Inhibition of basal ERK1/2 phosphorylation was determined by the following in vitro cellular proliferation assay, which comprises incubating cells with a compound of Formula I for 1 hour and quantifying the fluorescent pERK signal on fixed cells and normalizing to total ERK signal.

Materials and Methods: Malme-3M cells were obtained from ATCC and grown in RPMI-1640 supplemented with 10% fetal bovine serum. Cells were plated in 96-well plates at 24,000 cells/well and allowed to attach for 16-20 hours at 37° C., 5% CO₂. The media was removed, and DMSO-diluted compounds were added in RPMI-1640 at a final concentration of 1% DMSO. The cells were incubated with the compounds for 1 hour at 37° C., 5% CO₂. The cells were washed with PBS and fixed in 3.7% formaldehyde in PBS for 15 minutes. This was followed by washing in PBS/0.05% Tween20 and permeabilizing in −20° C. 100% MeOH for 15 minutes. Cells were washed in PBS/0.05% Tween20 then blocked in Odyssey blocking buffer (LI-COR Biosciences) for 1 hour. Antibodies to phosphorylated ERK (1:400, Cell Signaling #9106, monoclonal) and total ERK (1:400, Santa Cruz Biotechnology #sc-94, polyclonal) were added to the cells and incubated 16-20 hours at 4° C. After washing with PBS/0.05% Tween20, the cells were incubated with fluorescently-labeled secondary antibodies (1:1000 goat anti-rabbit IgG-IRDye800, Rockland and 1:500 goat anti-mouse IgG-Alexa Fluor 680, Molecular Probes) for an additional hour. Cells were then washed and analyzed for fluorescence at both wavelengths using the Odyssey Infrared Imaging System (LI-COR Biosciences). Phosphorylated ERK signal was normalized to total ERK signal.

The following compounds were tested in the above assay. Some compounds were prepared multiple times and tested in the above assay multiple times. The below data is representative of those tests:

| Example # | IC₅₀ (nM) |
|---|---|
| Example 1 | 68.8 |
| Example 5 | 13.9 |
| Example 10 | 26.7 |
| Example 11 | 9.55 |

| Example # | IC₅₀ (nM) |
|---|---|
| Example 17 | 664 |
| Example 22 | 42.75 |
| Example 24 | 381.5 |
| Example 34 | 125.95 |

### Intermediate Example A

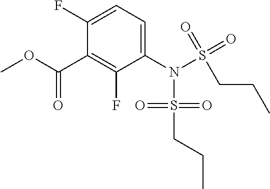

### methyl 2,6-difluoro-3-(N-(propylsulfonyl)propylsulfonamido)benzoate

Step A: A 1 L flask was charged with 2,6-difluoro-3-nitrobenzoic acid (17.0 g, 83.7 mmol) and MeOH (170 mL, 0.5M). The flask was placed in a cold water bath, and an addition funnel charged with a 2M solution of trimethylsilyl (“TMS”) diazomethane in hexanes (209 mL, 419 mmol) was attached to the flask. The TMS diazomethane solution was added slowly to the reaction flask over the course of 2 hours. A large excess of reagent was required in order for the reaction to reach completion as determined by the ceased evolution of N₂ upon further addition of reagent. The volatiles were removed in vacuo to afford methyl 2,6-difluoro-3-nitrobenzoate as a solid (18.2 g, 99%). The material was taken directly onto Step B.

Step B: 10% (wt.) Pd on activated carbon (4.46 g, 4.19 mmol) was added to a 1 L flask charged with methyl 2,6-difluoro-3-nitrobenzoate (18.2 g, 83.8 mmol) under a nitrogen atmosphere. EtOH (350 mL, 0.25 M) was added, and then H₂ was passed through the reaction mixture for 15 minutes. The reaction mixture was stirred under two H₂ balloons overnight. The following day the reaction mixture was re-flushed with fresh H₂ balloons and stirred an additional 4 hours. Upon consumption of the starting material and intermediate hydroxylamine as determined by TLC, N₂ gas was flushed through the reaction mixture. The mixture was then filtered through glass microfibre filter (“GF/F”) paper twice. The volatiles were removed to afford methyl 3-amino-2,6-difluorobenzoate as an oil (15.66 g, 99%). The material was taken directly onto the next step.

Step C: Propane-1-sulfonyl chloride (23.46 mL, 209.3 mmol) was slowly added to a solution of methyl 3-amino-2,6-difluorobenzoate (15.66 g, 83.7 mmol) and triethylamine (35.00 mL, 251.1 mmol) in CH₂Cl₂ (175 mL, 0.5M) maintained in a cool water bath. The reaction mixture was stirred for 1 hour at room temperature. Water (300 mL) was added and the organic layer was separated, washed with water (2×300 mL) and brine (200 mL), then dried (Na₂SO₄), filtered and concentrated to an oil. The crude product was purified by column chromatography, eluting with 15% ethyl acetate (“EtOAc”)/hexane. The isolated fractions were triturated with hexanes to afford methyl 2,6-difluoro-3-(N-(propylsulfonyl)propylsulfonamido)benzoate as a solid (24.4 g, 73% yield for 3 steps). ¹H NMR (400 MHz, CDCl₃) δ 7.52-7.45 (m, 1H), 7.08-7.02 (m, 1H), 3.97 (s, 3H), 3.68-3.59 (m, 2H), 3.53-3.45 (m, 2H), 2.02-1.89 (m, 4H), 1.10 (t, J=7.4 Hz, 6H). m/z (APCI-neg) M-(SO₂Pr)=292.2.

### Intermediate Example B

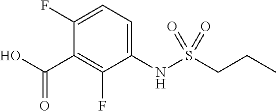

### 2,6-difluoro-3-(propylsulfonamido)benzoic acid

A 1N aqueous NaOH solution (150 mL, 150 mmol) was added to a solution of methyl 2,6-difluoro-3-(N-(propylsulfonyl)propylsulfonamido)benzoate (20.0 g, 50.1 mmol) in 4:1 THF/MeOH (250 mL, 0.2M). The reaction mixture was stirred at room temperature overnight. The majority of the organic solvents were then removed in vacuo (water bath temperature 35° C.). 1N HCl (150 mL) was slowly added to the mixture, and the resulting solid was filtered and rinsed with water (4×50 mL). The material was then washed with Et₂O (4×15 mL) to give 2,6-difluoro-3-(propylsulfonamido)benzoic acid as a solid (10.7 g, 77% yield). ¹H NMR (400 MHz, (CD₃)₂SO) δ 9.74 (s, 1H), 7.57-7.50 (m, 1H), 7.23-7.17 (m, 1H), 3.11-3.06 (m, 2H), 1.79-1.69 (m, 2H), 0.98 (t, J=7.4 Hz, 3H). m/z (APCI-neg) M−1=278.0.

### Intermediate Example C

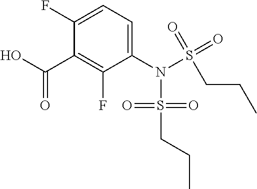

### 2,6-difluoro-3-(N-(propylsulfonyl)propylsulfonamido)benzoic acid

Propane-1-sulfonyl chloride (1.225 mL, 10.92 mmol) was added to a mixture of 3-amino-2,6-difluorobenzoic acid (0.573 g, 3.310 mmol), triethylamine (2.030 mL, 14.56 mmol) and CH₂Cl₂ (17 mL, 0.2M) cooled to 0° C. The reaction mixture was allowed to warm to room temperature and stirred for 1 hour. The mixture was then partitioned between saturated NaHCO₃ (100 mL) and ethyl acetate (75 mL). The aqueous layer was washed with ethyl acetate (50 mL) and then acidified with concentrated HCl to a pH of about 1. The acidified aqueous layer was extracted with ethyl acetate (2×50 mL), and the combined ethyl acetate extracts were dried (Na₂SO₄), filtered and concentrated. The resulting residue was triturated with hexanes to afford 2,6-difluoro-3-(N-(propylsulfonyl)propyl-sulfonamido)benzoic acid as a solid (0.948 g, 74% yield). ¹H NMR (400 MHz, (CD₃)₂SO) δ 7.90-7.84 (m, 1H), 7.39-7.34 (m, 1H), 3.73-3.58 (m, 4H), 1.88-1.74 (m, 4H), 1.01 (t, J=7.5 Hz, 6H). m/z (APCI-neg) M-(SO₂Pr)=278.1.

### Intermediate Example D

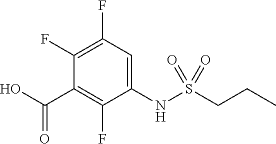

### 2,3,6-trifluoro-5-(propylsulfonamido)benzoic acid

2,3,6-Trifluoro-5-(propylsulfonamido)benzoic acid (8.5%) was prepared according to the general procedure of Intermediate Example C, substituting 3-amino-2,5,6-trifluorobenzoic acid for 3-amino-2,6-difluorobenzoic acid.

### Intermediate Example E

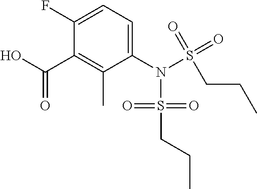

### 6-fluoro-2-methyl-3-(N-(propylsulfonyl)propylsulfonamido)benzoic acid

6-Fluoro-2-methyl-3-(N-(propylsulfonyl)propylsulfonamido)benzoic acid (11%) was prepared according to the general procedure of Intermediate Example C, substituting 3-amino-6-fluoro-2-methylbenzoic acid for 3-amino-2,6-difluorobenzoic acid.

### Intermediate Example F

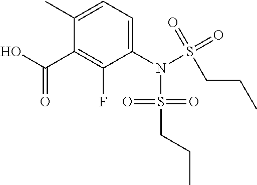

### 2-fluoro-6-methyl-3-(N-(propylsulfonyl)propylsulfonamido)benzoic acid

2-Fluoro-6-methyl-3-(N-(propylsulfonyl)propylsulfonamido)benzoic acid (3%) was prepared according to the general procedure of Intermediate Example C, substituting 3-amino-2-fluoro-6-methylbenzoic acid for 3-amino-2,6-difluorobenzoic acid.

### Intermediate Example G

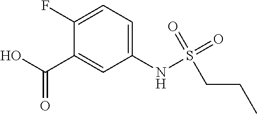

### 2-fluoro-5-(propylsulfonamido)benzoic acid

Propane-1-sulfonyl chloride (0.0871 mL, 0.774 mmol) was dissolved in 10% Na₂CO₃ (1.65 mL, 1.55 mmol) at room temperature. 5-Amino-2-fluorobenzoic acid (0.100 g, 0.645 mmol) was added and heated to 60° C. overnight. Propane-1-sulfonyl chloride (0.0871 mL, 0.774 mmol) was added again, and the reaction mixture was heated at 60° C. for another hour. The reaction mixture was cooled to room temperature, diluted with water, taken to a pH of 10 with 10% Na₂CO₃ and extracted with DCM (2×). The reaction mixture was then taken to a pH of 2 with 1N HCl, extracted with DCM (3×) and concentrated to a solid, 2-fluoro-5-(propylsulfonamido)benzoic acid (29%).

### Intermediate Example H

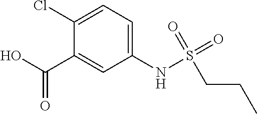

### 2-chloro-5-(propylsulfonamido)benzoic acid

2-Chloro-5-(propylsulfonamido)benzoic acid (14%) was prepared according to the general procedure for Intermediate Example G, substituting 5-amino-2-chlorobenzoic acid for 5-amino-2-fluorobenzoic acid.

### Intermediate Example I

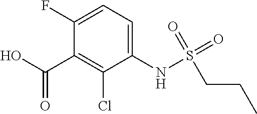

### 2-chloro-6-fluoro-3-(propylsulfonamido)benzoic acid

Step A: 2-Chloro-6-fluorobenzoic acid (2.00 g, 11.5 mmol) was dissolved in sulfuric acid (20 mL) and cooled to 0° C. Nitric acid (0.529 mL, 12.6 mmol) was added, and the reaction mixture was warmed to room temperature for one hour. The reaction mixture was diluted with water, and the aqueous portion was extracted with DCM (3×), dried over Na₂SO₄, concentrated to a solid, 2-chloro-6-fluoro-3-nitrobenzoic acid (97%), which was used directly in the next step without further purification.

Step B: 2-Chloro-6-fluoro-3-nitrobenzoic acid (0.100 g, 0.455 mmol) and Zn dust (0.298 g, 4.55 mmol) were taken up in THF (4 mL) and saturated aqueous NH₄Cl (2 mL) and stirred at room temperature overnight. The reaction mixture was filtered through Celite, concentrated to a solid, and dissolved in water. The pH was adjusted to 2 with 1N HCl, and the aqueous portion was extracted with DCM (3×). The organic portion was dried over Na₂SO₄ and concentrated to a solid, 3-amino-2-chloro-6-fluorobenzoic acid (49%), which was used directly in the next step without further purification.

Step C: 2-Chloro-6-fluoro-3-(propylsulfonamido)benzoic acid (13%) was prepared according to the general procedure for Intermediate Example G, substituting 3-amino-2-chloro-6-fluorobenzoic acid for 5-amino-2-fluorobenzoic acid.

### Intermediate Example J

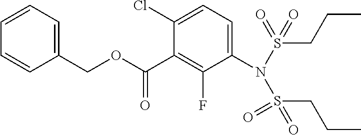

### benzyl 6-chloro-2-fluoro-3-(N-(propylsulfonyl)propylsulfonamido)benzoate

Step A: A flame dried flask equipped with a stir bar and rubber septum was charged with 4-chloro-2-fluoroaniline (5.00 g, 34.35 mmol) and dry THF (170 mL). This solution was chilled to −78° C., and n-BuLi (14.7 mL, 1.07 eq. of 2.5M solution in hexanes) was then added over a 15 minute period. This mixture was stirred at −78° C. for 20 minutes, and then a THF solution (25 mL) of 1,2-bis(chlorodimethylsilyl)ethane (7.76 g, 1.05 eq.) was added slowly (over a 10 minute period) to the reaction mixture. This was stirred for 1 hour, and then 2.5M n-BuLi in hexanes (15.11 mL, 1.1 eq.) was added slowly. After allowing the mixture to warm to room temperature for one hour, the mixture was chilled back to −78° C. A third allotment of n-BuLi (15.66 mL, 1.14 eq.) was added slowly, and the mixture was stirred at −78° C. for 75 minutes. Benzyl chloroformate (7.40 g, 1.2 eq.) was then added slowly, and the mixture was stirred at −78° C. for one hour. The cooling bath was then removed. The mixture was allowed to warm for 30 minutes and then quenched with water (70 mL) and concentrated HCl (25 mL). The mixture was allowed to continue to warm to room temperature. The mixture was then extracted with EtOAc. The extracts were washed twice with a saturated Na₂HCO₃ solution, once with water, dried over sodium sulfate and concentrated. The resulting residue was flashed on a 65 Biotage (30% ethyl acetate/hexane) to produce benzyl 3-amino-6-chloro-2-fluorobenzoate (4.3 g, 45%) as an oil. ¹H NMR ((CD₃)₂SO, 400 MHz) δ 7.37-7.48 (m, 5H), 7.07 (dd, 1H, J=8, 2), 6.87 (t, 1H, J=8), 5.61 (br s, 2H), 5.40 (s, 2H).

Step B: Benzyl 3-amino-6-chloro-2-fluorobenzoate (4.3 g, 15.37 mmol) was dissolved in dry dichloromethane (270 mL). Triethylamine (5.36 mL, 2.5 eq.) was added, and the mixture was chilled to 0° C. Propane-1-sulfonyl chloride (3.63 mL, 32.3 mmol, 2.1 eq.) was then added via syringe, and a precipitate resulted. Once the addition was complete, the mixture was allowed to warm to room temperature, and the starting material was consumed as determined by TLC (3:1 hexane:ethyl acetate). The mixture was then diluted with dichloromethane (200 mL), washed with 2M aqueous HCl (2×100 mL), saturated Na₂HCO₃ solution, dried over sodium sulfate and concentrated. The resulting residue was purified on a 65 Biotage chromatography system (40% ethyl acetate/hexane) to produce benzyl 6-chloro-2-fluoro-3-(N-(propylsulfonyl)propylsulfonamido)benzoate (5.5 g, 72%) as an oil that slowly solidified upon standing. ¹H NMR (CDCl₃, 400 MHz) δ 7.28-7.45 (m, 7H), 5.42 (s, 2H), 3.58-3.66 (m, 2H), 3.43-3.52 (m, 2H), 1.08 (t, 6H, J=8).

### Intermediate Example K

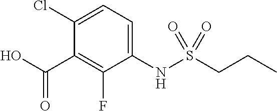

### 6-chloro-2-fluoro-3-(propylsulfonamido)benzoic acid

Benzyl 6-chloro-2-fluoro-3-(N-(propylsulfonyl)propylsulfonamido)benzoate (5.4 g, 10.98 mmol) was dissolved in THF (100 mL) and 1M aqueous KOH (100 mL). This mixture was refluxed for 16 hours and then allowed to cool to room temperature. The mixture was then acidified to a pH of 2 with 2M aqueous HCl and extracted with EtOAc (2×). The extracts were washed with water, dried over sodium sulfate and concentrated to a solid that was triturated with hexanes/ether to give 6-chloro-2-fluoro-3-(propylsulfonamido)benzoic acid (2.2 g, 68%) as a solid. ¹H NMR ((CD₃)₂SO, 400 MHz) δ 9.93 (s, 1H), 7.49 (t, 1H, J=8), 7.38 (dd, 1H, J=8, 2), 3.11-3.16 (m, 2H), 1.68-1.78 (m, 2H), 0.97 (t, 3H, J=8).

### Intermediate Example L

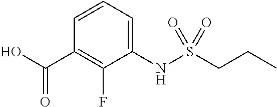

### 2-fluoro-3-(propylsulfonamido)benzoic acid

6-Chloro-2-fluoro-3-(propylsulfonamido)benzoic acid (0.5 g, 1.69 mmol) was dissolved in methanol (15 mL), and Peariman's catalyst (one weight equivalent, 0.5 g, 20% Pd(OH)₂ on carbon, 50% by weight water) was added. This mixture was subjected to a balloon of hydrogen for 3 hours and then filtered through GF/F filter paper. The filtrate was concentrated to 2-fluoro-3-(propylsulfonamido)benzoic acid (396 mg, 90%) as a solid. MS (M-H+) 262. ¹H NMR ((CD₃)₂SO, 400 MHz) δ 13.36 (s, 1H), 9.76 (s, 1H), 7.58-7.70 (m, 2H), 7.26 (t, 1H, J=8), 3.10 (t, 2H, J=8), 1.69-1.80 (m, 2H), 0.98 (t, 3H, J=8).

### Intermediate Example M

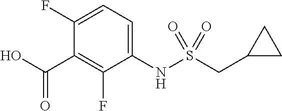

### 3-(cyclopropylmethylsulfonamido)-2,6-difluorobenzoic acid

Step A: Cyclopropylmethanesulfonyl chloride (1.27 g, 8.20 mmol) was added to a mixture of 3-amino-2,6-difluorobenzoic acid (0.430 g, 2.48 mmol), triethylamine (1.52 mL, 10.9 mmol) and CH₂Cl₂ (12 mL, 0.2M) cooled to 0° C. The reaction mixture was allowed to warm to room temperature and stirred for 1 hour. The mixture was then partitioned between saturated NaHCO₃ (75 mL) and ethyl acetate (50 mL). The aqueous layer was washed with ethyl acetate (50 mL) and then acidified to a pH of 1 with concentrated HCl. The acidified aqueous layer was extracted twice with ethyl acetate (2×50 mL), and the combined ethyl acetate extracts were dried (Na₂SO₄), filtered and concentrated to provide crude 3-(1-cyclopropyl-N-(cyclopropylmethylsulfonyl)methylsulfonamido)-2,6-difluorobenzoic acid (380 mg, 37%).

Step B: A solution of 1N NaOH (2.78 mL, 2.78 mmol) was added to a solution of crude 3-(1-cyclopropyl-N-(cyclopropylmethyl sulfonyl)methylsulfonamido)-2,6-difluorobenzoic acid (380 mg, 0.928 mmol) in 4:1 THF/MeOH (5 mL, 0.2M). The reaction mixture was stirred at room temperature for 1 hour, after which most of the organic solvents were removed. 1N HCl (3 mL) was slowly added to the mixture to acidify to a pH of 1. The acidified aqueous layer was extracted with ethyl acetate (75 mL). The ethyl acetate extract was washed with water (2×20 mL), brine (20 mL), dried (Na₂SO₄), filtered and concentrated. Trituration of the residue with Et₂O afforded 3-(cyclopropylmethylsulfonamido)-2,6-difluorobenzoic acid as a solid (139 mg, 51%). ¹H NMR (400 MHz, (CD₃)₂SO) δ 9.76 (s, 1H), 7.60-7.54 (m, 1H), 7.22-7.16 (m, 1H), 3.10 (d, J=7.0 Hz, 2H), 1.10-0.99 (m, 1H), 0.58-0.53 (m, 2H), 0.36-0.31 (m, 2H); m/z (APCI-neg) M−1=289.9.

### Intermediate Example N

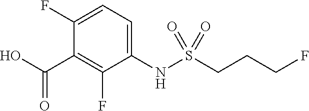

### 2,6-difluoro-3-(3-fluoropropylsulfonamido)benzoic acid

Methyl 2,6-difluoro-3-(N-(3-fluoropropylsulfonyl)-3-fluoropropylsulfonamido)benzoate was made according to the general procedure for Intermediate Example A, substituting 3-fluoropropyl sulfonyl chloride for propane-1-sulfonyl chloride. ¹H NMR (400 MHz, DMSO-d₆) δ 8.05-7.99 (m, 1H), 7.44 (t, 1H), 4.62 (t, 2H), 4.50 (t, 2H), 3.93 (s, 3H), 3.89-3.74 (m, 4H), 2.26-2.11 (m, 4H).

2,6-Difluoro-3-(3-fluoropropylsulfonamido)benzoic acid was prepared according to the general procedure for Intermediate Example B, substituting methyl 2,6-difluoro-3-(N-(3-fluoropropylsulfonyl)-3-fluoropropylsulfonamido)benzoate for methyl 2,6-difluoro-3-(N-(propylsulfonyl)-propylsulfonamido)benzoate. ¹H NMR (500 MHz, (CD₃)₂SO) δ 14.05 (br s, 1H), 9.71 (s, 1H), 7.56-7.50 (m, 1H), 7.20 (t, 1H), 3.12-3.08 (m, 2H), 1.73-1.66 (m, 2H), 1.39 (sx, 2H), 0.87 (t, 3H).

### Intermediate Example 0

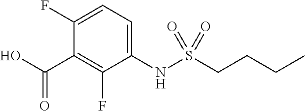

### 3-(butylsulfonamido)-2,6-difluorobenzoic acid

Methyl 2,6-difluoro-3-(N-(butylsulfonyl)-butylsulfonamido)benzoate was made according to the general procedure for Intermediate Example A, substituting butane-1-sulfonyl chloride for propane-1-sulfonyl chloride. ¹H NMR (500 MHz, DMSO-d₆) δ 7.99-7.94 (m, 1H), 7.42 (t, 1H), 3.92 (s, 3H), 3.74-3.62 (m, 4H), 1.81-1.68 (m, 4H), 1.42 (sx, 4H), 0.89 (t, 6H).

3-(Butylsulfonamido)-2,6-difluorobenzoic acid was prepared according to the general procedure for Intermediate Example B, substituting methyl 2,6-difluoro-3-(N-(butylsulfonyl)-butylsulfonamido)benzoate for methyl 2,6-difluoro-3-(N-(propylsulfonyl)-propylsulfonamido)benzoate. ¹H NMR (400 MHz, (CD₃)₂SO) δ 14.05 (br s, 1H), 9.71 (s, 1H), 7.56-7.50 (m, 1H), 7.20 (t, 1H), 3.12-3.08 (m, 2H), 1.73-1.66 (m, 2H), 1.39 (sx, 2H), 0.87 (t, 3H).

### Intermediate Example P

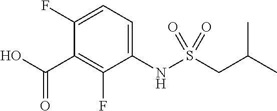

### 2,6-difluoro-3-(2-methylpropylsulfonamido)benzoic acid

Methyl-2,6-difluoro-3-(N-(2-methylpropylsulfonyl)-2-methylpropyl-sulfonamido)benzoate was made according to the general procedure for Intermediate Example A, substituting 2-methylpropyl sulfonyl chloride for propane-1-sulfonyl chloride. m/z (LC-MS) M+1=428.4.

2,6-Difluoro-3-(2-methylpropylsulfonamido)benzoic acid was prepared according to the general procedure for Intermediate Example B, substituting methyl-2,6-difluoro-3-(N-(2-methylpropylsulfonyl)-2-methylpropylsulfonamido)benzoate for methyl 2,6-difluoro-3-(N-(propylsulfonyl)-propylsulfonamido)benzoate. ¹H NMR (400 MHz, (CD₃)₂SO) δ 14.01 (s, 1H), 9.71 (s, 1H), 7.56 (dd, 1H), 7.22 (dd, 1H), 3.02 (d, 2H), 2.18-2.15 (m, 1H), 1.03 (d, 6H); m/z (LC-MS) M+1=294.3.

### Intermediate Example Q

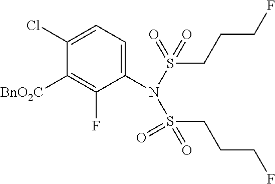

### benzyl 6-chloro-2-fluoro-3-(3-fluoro-N-(3-fluoropropylsulfonyl)propyl sulfonamido)benzoate

Benzyl 6-chloro-2-fluoro-3-(3-fluoro-N-(3-fluoropropylsulfonyl)propylsulfonamido)benzoate (92%) was prepared according to the general procedure for Intermediate Example J, Step B substituting 3-fluoropropane-1-sulfonyl chloride for propane-1-sulfonyl chloride.

### Intermediate Example R

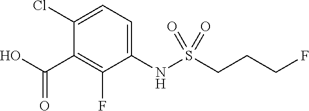

### 6-chloro-2-fluoro-3-(3-fluoropropylsulfonamido)benzoic acid

6-Chloro-2-fluoro-3-(3-fluoropropylsulfonamido)benzoic acid (71%) was prepared according to the general procedure for Intermediate Example K substituting benzyl 6-chloro-2-fluoro-3-(3-fluoro-N-(3-fluoropropylsulfonyl)propylsulfonamido)benzoate for benzyl 6-chloro-2-fluoro-3-(N-(propylsulfonyl)propylsulfonamido)benzoate.

### Intermediate Example S

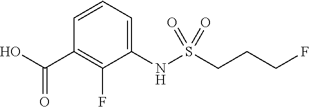

### 2-fluoro-3-(3-fluoropropylsulfonamido)benzoic acid

2-Fluoro-3-(3-fluoropropylsulfonamido)benzoic acid (81%) was prepared according to the general procedure for Intermediate Example L substituting 6-chloro-2-fluoro-3-(3-fluoropropylsulfonamido)benzoic acid for 6-chloro-2-fluoro-3-(propylsulfonamido)benzoic acid.

### Intermediate Example T

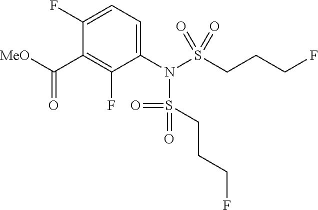

### methyl 2,6-difluoro-3-(3-fluoro-N-(3-fluoropropylsulfonyl)propylsulfonamido)benzoate

3-Fluoropropane-1-sulfonyl chloride (14.3 mL, 129 mmol) was slowly added to a solution of methyl 3-amino-2,6-difluorobenzoate (24.1 g, 129 mmol) and pyridine (31.2 mL, 386 mmol) in CH₂Cl₂ (360 mL). The reaction mixture was stirred for over two days at room temperature. The reaction mixture was diluted with methylene chloride. The reaction mixture was then washed with an aqueous solution of saturated sodium bicarbonate, 1N HCl, and brine, then dried (Na₂SO₄), filtered and concentrated to an oil to give methyl 2,6-difluoro-3-(3-fluoro-N-(3-fluoropropylsulfonyl)propylsulfonamido)benzoate (38.1 g). ¹H NMR (400 MHz, CDCl₃, ppm) 7.69 (dt, 1H), 7.00 (dt, 1H), 6.55 (s, 1H), 4.56 (dd, 2H), 3.28-3.17 (m, 2H), 2.32-2.15 (m, 2H).

### Intermediate Example U

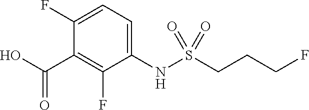

### 2,6-difluoro-3-(3-fluoropropylsulfonamido)benzoic acid

2,6-Difluoro-3-(N-(3-fluoropropylsulfonyl)propylsulfonamido)benzoate (38 g, 120 mmol) was dissolved in 5:2 THF/MeOH (250 mL), and a solution of lithium hydroxide (8.77 g, 366 mmol) in water (50 mL) was added. The reaction mixture was stirred at room temperature for four hours. The majority of the organic solvents were then removed in vacuo. 2.5N HCl (500 mL) was slowly added to the mixture, and the resulting solid was filtered and rinsed with cold ether to give 2,6-difluoro-3-(3-fluoropropylsulfonamido)benzoic acid as a solid (29.3 g, 81% yield). ¹H NMR (400 MHz, CDCl₃ ppm) 9.85 (s, 1H), 7.54 (dt, 1H), 7.21 (dt, 1H), 4.54 (td, 2H), 2.20-2.00 (m, 2H), 3.24-3.18 (m, 2H).

### Intermediate Example V

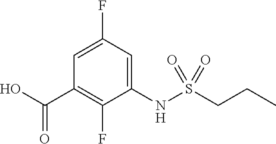

### 2,5-difluoro-3-(propylsulfonamido)benzoic acid

Step A: 2,5-Difluorobenzoic acid (2.01 g, 9.90 mmol, 31.3% yield) was dissolved in concentrated sulfuric acid (25 mL) and cooled to 0° C. Nitric Acid (1.46 mL, 34.8 mmol) was added, and the reaction mixture was stirred at room temperature for one hour. The solution was extracted with DCM (3×), and the combined organic layers were dried over sodium sulfate and concentrated. The residue was purified by column chromatography (1:1 hexanes:1% HCOOH/EtOAc) giving 2,5-difluoro-3-nitrobenzoic acid (2.01 g, 31.3%) as a solid.

Step B: 2,5-Difluoro-3-nitrobenzoic acid (2.00 g, 9.847 mmol) was dissolved in MeOH (60 mL). TMSCl (6.220 mL, 49.24 mmol) was added, and the reaction mixture was stirred at reflux for 4 hours. The reaction mixture was concentrated to about 20 mL, and the crystals produced were filtered and dried under high vacuum providing methyl 2,5-difluoro-3-nitrobenzoate (1.55 g, 72.4%) as a crystalline solid.

Step C: Methyl 3-amino-2,5-difluorobenzoate (96.5%) was prepared according to the general procedure for Intermediate Example A, Step B, substituting methyl 2,5-difluoro-3-nitrobenzoate for methyl 2,6-difluoro-3-nitrobenzoate.

Step D: Methyl 2,5-difluoro-3-(N-(propylsulfonyl)propylsulfonamido) benzoate was prepared according to the general procedure for Intermediate Example A, Step C, substituting methyl 3-amino-2,5-difluorobenzoate for methyl 3-amino-2,6-difluorobenzoate.

Step E: 2,5-Difluoro-3-(propylsulfonamido)benzoic acid (83.8%, two steps) was prepared according to the general procedure for Intermediate Example B substituting methyl 2,5-difluoro-3-(N-(propylsulfonyl)propylsulfonamido)benzoate for methyl 2,6-difluoro-3-(N-(propylsulfonyl)propylsulfonamido)benzoate. ¹H NMR (400 MHz, (CD₃)₂SO) δ 13.67 (br s, 1H), 10.07 (s, 1H), 7.46-7.50 (m, 1H), 7.38-7.42 (m, 1H), 3.17-3.21 (m, 2H), 1.70-1.76 (m, 2H), 0.95-0.99 (m, 3H); m/z (APCI-neg) M−1=278.1.

### Intermediate Example W

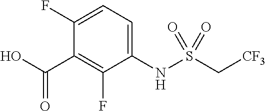

### 2,6-difluoro-3-(2,2,2-trifluoroethylsulfonamido)benzoic acid

Step A: 2,2,2-Trifluoroethyl-sulfonyl chloride (459 mL, 4.15 mmol) was slowly added to a solution of methyl 3-amino-2,6-difluorobenzoate (311 g, 1.66 mmol) and pyridine (0.806 mL, 9.97 mmol) in dichloromethane (8.92 mL, 139 mmol), while applying external cooling using an acetone dry ice bath. The reaction mixture was stirred for 45 minutes, and the dry ice bath was removed. The reaction mixture was kept stirring for another hour. The mixture was diluted with EtOAc (100 mL), washed with water (2×25 mL) and brine (25 mL), dried (Na₂SO₄), filtered, and then concentrated to an oil. The crude product was purified by column chromatography, eluting with 15% EtOAc/hexane to afford methyl 2,6-difluoro-3-(2-trifluoroethylsulfonamido) benzoate as a solid (513 mg, 92.6% yield). ¹H NMR (400 MHz, (CD₃)₂SO) δ 8.10-8.01 (m, 1H), 7.48 (t, 1H), 4.68 (s, 2H), 4.58 (s, 2H), 3.98 (s, 3H).

Step B: 2,6-Difluoro-3-(2-trifluoroethylsulfonamido)benzoic acid was prepared according to the general procedure for Intermediate Example B, substituting methyl 2,6-difluoro-3-(2-trifluoroethylsulfonamido) benzoate for methyl 2,6-difluoro-3-(N-(propylsulfonyl)-propylsulfonamido)benzoate. ¹H NMR (500 MHz, (CD₃)₂SO) δ 14.08 (br s, 1H), 9.75 (s, 1H), 7.58-7.52 (m, 1H), 7.25 (t, 1H), 3.15-3.11 (s, 2H).

### Intermediate Example X

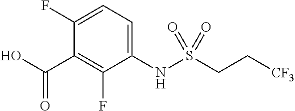

### 2,6-difluoro-3-(3,3,3-trifluoropropyl sulfonamido)benzoic acid

Step A: Methyl 2,6-difluoro-3-(N-(3,3,3-trifluoropropyl sulfonyl)-3,3,3-trifluoropropyl-sulfonamido) benzoate was made according to the general procedure for Intermediate Example A, substituting 3,3,3-trifluoropropyl sulfonyl chloride for propane-1-sulfonyl chloride. ¹H NMR (400 MHz, (CD₃)₂SO) δ 8.05-7.99 (m, 1H), 7.44 (t, 1H), 4.62 (t, 2H), 4.50 (t, 2H), 3.93 (s, 3H), 3.89-3.74 (m, 4H), 2.26-2.11 (m, 4H).

Step B: 2,6-Difluoro-3-(3,3,3-trifluoropropylsulfonamido)benzoic acid was prepared according to the general procedure for Intermediate Example B, substituting methyl 2,6-difluoro-3-(N-(3,3,3-trifluoropropylsulfonyl)-3,3,3-trifluoropropylsulfonamido)benzoate for methyl 2,6-difluoro-3-(N-(propylsulfonyl)-propylsulfonamido)benzoate. ¹H NMR (500 MHz, (CD₃)₂SO) δ 14.05 (br s, 1H), 9.71 (s, 1H), 7.56-7.50 (m, 1H), 7.20 (t, 1H), 3.12-3.08 (m, 2H), 1.73-1.66 (m, 2H).

### Intermediate Example Y

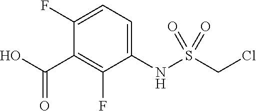

### 2,6-difluoro-3-(2-chloromethylsulfonamido)benzoic acid

Step A: Methyl 2,6-difluoro-3-(N-(2-chloromethylsulfonyl)-2-chloromethyl-sulfonamido)benzoate was made according to the general procedure for Intermediate Example A, substituting 2-chloromethyl sulfonyl chloride for propane-1-sulfonyl chloride. ¹H NMR (400 MHz, (CD₃)₂SO) δ 8.08-7.97 (m, 1H), 7.45 (t, 1H), 4.65 (s, 2H), 4.55 (s, 2H), 4.02 (s, 3H).

Step B: 2,6-Difluoro-3-(2-chloromethylsulfonamido)benzoic acid was prepared according to the general procedure for Intermediate Example B, substituting methyl 2,6-difluoro-3-(N-(2-chloromethylsulfonyl)-2-chloromethylsulfonamido)benzoate for methyl 2,6-difluoro-3-(N-(propylsulfonyl)-propylsulfonamido)benzoate. ¹H NMR (500 MHz, (CD₃)₂SO) δ 14.10 (br s, 1H), 9.78 (s, 1H), 7.62-7.56 (m, 1H), 7.28 (t, 1H), 3.19-3.15 (s, 2H).

### Intermediate Example Z

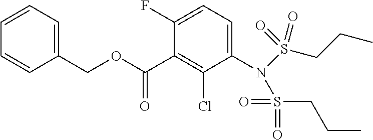

### benzyl 2-chloro-6-fluoro-3-(N-(propylsulfonyl)propylsulfonamido)benzoate

Step A: Benzyl 3-amino-2-chloro-6-fluorobenzoate (56%) was prepared according to the general procedure for Intermediate Example J, substituting 2-chloro-4-fluoroaniline for 4-chloro-2-fluoroaniline. ¹H NMR (400 MHz, d₆-DMSO) δ 7.48-7.32 (m, 5H), 7.11-7.05 (t, 1H), 6.94-6.89 (q, 1H), 5.53-5.49 (s, 2H), 5.41-5.39 (s, 2H).

Step B: Benzyl 3-amino-2-chloro-6-fluorobenzoate (330 mg, 1.2 mmol) was dissolved in dry dichloromethane (11.8 mL). Triethylamine (0.494 mL, 3.54 mmol) was added, and the mixture was chilled to 0° C. Propane-1-sulfonyl chloride (0.332 mL, 2.95 mmol) was then added via syringe. Once the addition was complete, the mixture was allowed to warm to ambient temperature and stir for 16 hours. The mixture was diluted with dichloromethane (11 mL) and washed with water (2×50 mL) and brine (25 mL), dried over sodium sulfate, and concentrated. The resulting residue was applied directly to a silica gel column and eluted with a gradient (5% to 40%) of ethyl acetate-hexanes to provide benzyl 2-chloro-6-fluoro-3-(N-(propylsulfonyl)propylsulfonamido)benzoate (413 mg, 0.840 mmol, 71.1% yield). ¹H NMR (400 MHz, (CD₃)₂SO) δ 8.00-7.94 (q, 1H), 7.59-7.52 (t, 1H), 7.50-7.35 (m, 5H), 5.48-5.44 (s, 2H), 3.80-3.60 (m, 4H), 1.89-1.75 (m, 4H), 1.05-0.98 (t, 6H).

### Intermediate Example AB

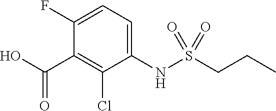

### 2-chloro-6-fluoro-3-(propylsulfonamido)benzoic acid

Step A: Benzyl 2-chloro-6-fluoro-3-(N-(propylsulfonyl)propylsulfonamido)benzoate (413.2 mg, 0.840 mmol) was dissolved in THF (8.4 mL) and 2.0M aqueous LiOH (1.26 mL). The mixture was refluxed for 16 hours and then allowed to cool to ambient temperature. The mixture was acidified to a pH of 0 with 1.0M HCl (5.0 mL) and then adjusted to a pH of 4 using saturated sodium bicarbonate. The mixture was extracted with EtOAc (2×). The extracts were washed with water (2×) and brine (1×), dried over sodium sulfate and concentrated to afford benzyl 2-chloro-6-fluoro-3-(propylsulfonamido)benzoate (174.5 mg, 0.452 mmol, 53.9% yield). MS (APCI-neg) m/z=384.1 (M-H).

Step B: Benzyl 2-chloro-6-fluoro-3-(propylsulfonamido)benzoate (174.5 mg, 0.4523 mmol) was dissolved in 3:1 dioxane:water (7.5 mL) and treated with barium hydroxide (100.7 mg, 0.5879 mmol). The reaction mixture was heated to 80° C. for 16 hours and then allowed to cool to ambient temperature. The mixture was acidified to a pH of 0 with concentrated HCl. The reaction mixture was allowed to stir for 10 minutes, after which the pH was adjusted to a pH of 4 using saturated sodium bicarbonate. The mixture was extracted with EtOAc (2×). The extracts were washed with water (2×) and brine (1×), dried over sodium sulfate, and concentrated to afford 2-chloro-6-fluoro-3-(propylsulfonamido)benzoic acid (75.7 mg, 0.256 mmol, 56.6% yield). MS (APCI-neg) m/z=293.9 (M-H).

### Intermediate Example AC

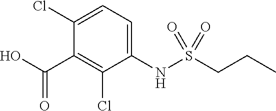

2,6-dichloro-3-(propylsulfonamido)benzoic acid

Step A: 2,6-Dichloro-3-nitrobenzoic acid (2.13 g, 9.03 mmol) was dissolved in 2:1 THF:saturated aqueous NH₄Cl and cooled to 0° C. The mixture was treated with zinc (11.8 g, 181 mmol). The reaction mixture was allowed to warm to ambient temperature and stir for 24 hours. The reaction mixture was filtered through GF/F paper while rinsing with THF. The mixture was acidified to a pH of 1 using 1.0M HCl and extracted with 15% 2-propanol:DCM (3 X). The extracts were washed with water and brine, dried over sodium sulfate and concentrated to afford 3-amino-2,6-dichlorobenzoic acid (1.40 g, 6.82 mmol, 75.5% yield). MS (APCI-neg) m/z=203.6 (M-H).

Step B: 3-Amino-2,6-dichlorobenzoic acid (1.40 g, 6.82 mmol) was dissolved in dry dichloromethane (66.7 mL). Triethylamine (4.09 mL, 29.4 mmol) was added, and the mixture was chilled to 0° C. Propane-1-sulfonyl chloride (2.48 mL, 22 mmol) was then added via syringe. Once the addition was complete, the mixture was allowed to warm to ambient temperature and stir for 1 hour. The mixture was concentrated in vacuo and diluted with diethyl ether. The mixture was washed with 0.25M NaOH (80 mL), and the aqueous layer was acidified to a pH of 1 using 1.0M HCl. The aqueous layer was extracted with 15% 2-propanol:DCM (2×300 mL). The organic layer was collected, dried over sodium sulfate, and concentrated to afford 2,6-dichloro-3-(propylsulfonamido)benzoic acid (1.55 g, 4.96 mmol, 74.4% yield). ¹H NMR (400 MHz, (CD₃)₂SO) δ 9.77-9.75 (s, 1H), 7.84-7.80 (d, 1H), 7.71-7.68 (d, 1H), 3.82-3.72 (m, 2H), 1.89-1.70 (m, 2H), 1.05-1.03 (m, 3H).

### Intermediate Example AD

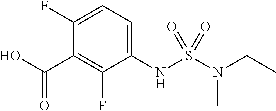

### 3-(N-ethyl-N-methylsulfamoylamino)-2,6-difluorobenzoic acid

Step A: A solution of triethylamine (0.260 mL, 1.85 mmol) and methyl 3-amino-2,6-difluorobenzoate (0.257 mL, 1.85 mmol) was added dropwise to sulfuryl dichloride (0.156 mL, 1.85 mmol) in DCM (3 mL) at −78° C. After 2 hours, N-methylethanamine (0.304 mL, 3.70 mmol) was added, and the reaction mixture was allowed to warm to room temperature overnight. The solvent was concentrated under reduced pressure, and the residue was taken up in NaOH (2 mL, 1M) and washed with EtOAc. The aqueous pH was lowered to below 3 and, the mixture was extracted with EtOAc (3×5 mL) The combined organic layers were dried over sodium sulfate, decanted and concentrated under reduced pressure. The residue was purified via silica gel chromatography eluting with 7:3 hexane:EtOAc to afford methyl 3-(N-ethyl-N-methylsulfamoylamino)-2,6-difluorobenzoate (0.280 g, 49.0% yield).

Step B: NaOH (0.908 mL, 1.82 mmol) was added to methyl 3-(N-ethyl-N-methylsulfamoylamino)-2,6-difluorobenzoate (0.280 g, 0.908 mmol) in THF:MeOH (3:2; 5 mL) The mixture was warmed to 60° C. for 16 hours. The cooled mixture was concentrated under reduced pressure, and the residue was taken up in 1M NaOH (4 mL) and washed with EtOAc. The aqueous pH was lowered to below 3, and the mixture was extracted with EtOAc (3×6 mL) to provide 3-(N-ethyl-N-methylsulfamoylamino)-2,6-difluorobenzoic acid (222 mg, 83% yield).

### Example 1

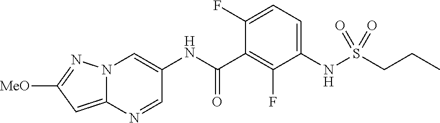

### 2,6-difluoro-N-(2-methoxypyrazolo[1,5-a]pyrimidin-6-yl)-3-(propylsulfonamido)benzamide

Step A: A suspension of 3-methoxy-1H-pyrazol-5-amine (0.21 g, 1.89 mmol, prepared as described in JP 01013072) and sodium nitromalonaldehyde monohydrate (0.31 g, 1.98 mmol) in acetic acid (6 mL) was stirred at room temperature for 1 hour. The reaction mixture was diluted with water (100 mL) and the resulting solids (0.198 g, 0.102 mmol, 54% yield) were collect by filtration and dried under vacuo to provide 2-methoxy-6-nitropyrazolo[1,5-a]pyrimidine. ¹H NMR (400 MHz, (CD₃)₂SO) δ 10.06 (d, J=2.4 Hz, 1H), 9.13 (d, J=2.4 Hz, 1H), 6.54 (s, 1H), 4.04 (s, 3H); m/z (APCI-nega) M−1=193.8.

Step B: 10% wt Pd/C (0.109 g, 0.102 mmol) was added to a solution of 2-methoxy-6-nitropyrazolo[1,5-a]pyrimidine (0.198 g, 1.02 mmol) in a mixture of ethyl acetate/MeOH (1:1, 20 mL) The reaction mixture was purged with N₂ for 10 minutes and then placed under a balloon of H₂ for 2 hours. The Pd/C was removed by filtration, and the filtrate was concentrated. The crude product was purified by flash chromatography, eluting with hexanes/ethyl acetate (1:4) to give 2-methoxypyrazolo[1,5-a]pyrimidin-6-amine (0.037 g, 0.022 mmol, 22% yield) as a solid. ¹H NMR (400 MHz, CD₃OD) δ 8.17 (s, 1H), 8.08 (s, 1H), 5.87 (s, 1H), 3.93 (s, 3H); m/z (APCI-pos) M+1=165.1.

Step C: 2-Methoxypyrazolo[1,5-a]pyrimidin-6-amine (37 mg, 0.225 mmol) was dissolved in DMF (5 mL) and sequentially treated with 2,6-difluoro-3-(propylsulfonamido)benzoic acid (66 mg, 0.237 mmol), anhydrous 1-ethyl-(3-dimethylaminopropyl)carbodiimide hydrochloride (67 mg, 0.24 mmol), and 1-hydroxybenzotriazole (9 mg, 0.068 mmol) at ambient temperature. After 16 hours, the reaction mixture was diluted with EtOAc and washed with water (4×), sodium bicarbonate (2×), brine (1×), dried over sodium sulfate and concentrated. Silica gel chromatography eluting with eluting with hexanes/ethyl acetate (2:1) gave 2,6-difluoro-N-(2-methoxypyrazolo[1,5-a]pyrimidin-6-yl)-3-(propylsulfonamido)benzamide (50 mg, 0.118 mmol, 52% yield) as a solid. ¹H NMR (400 MHz, CD₃OD) δ 9.43 (s, 1H), 8.44 (s, 1H), 7.66 (m, 1H), 7.15 (m, 1H), 6.07 (s, 1H), 4.01 (s, 3H), 3.12 (m, 2H), 1.87 (m, 2H), 1.06 (t, J=8.0 Hz, 3H); m/z (APCI-nega) M−1=424.0.

### Example 2

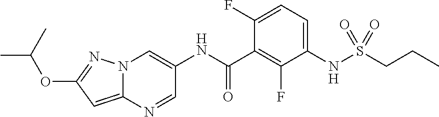

2,6-difluoro-N-(2-isopropoxypyrazolo[1,5-a]pyrimidin-6-yl)-3-(propylsulfonamido)benzamide

Step A: A mixture of 2-cyanoacetohydrazide (1.00 g, 10.09 mmol), i-PrOH (10 mL) and methanesulfonic acid (1.37 mL, 21.2 mmol) was heated to 82° C. for 40 hours. The mixture was then partitioned between 2N NaOH (50 mL) and DCM (200 mL). The organic layer was dried (Na₂SO₄), filtered and concentrated to afford 3-isopropoxy-1H-pyrazol-5-amine as a solid (80 mg, 6% yield).

Step B: 2-Isopropoxy-6-nitropyrazolo[1,5-a]pyrimidine (0.170 g, 93% yield) was prepared according to the general procedure in Example 1, Step A, substituting 3-isopropoxy-1H-pyrazol-5-amine for 3-methoxy-1H-pyrazol-5-amine.

Step C: 2-Isopropoxypyrazolo[1,5-a]pyrimidin-6-amine (46 mg, 31% yield) was prepared according to the general procedure in Example 1, Step B, substituting 2-isopropoxy-6-nitropyrazolo[1,5-a]pyrimidine for 2-methoxy-6-nitropyrazolo[1,5-a]pyrimidine.

Step D: 2,6-Difluoro-N-(2-isopropoxypyrazolo[1,5-a]pyrimidin-6-yl)-3-(propylsulfonamido)benzamide (68 mg, 68% yield) was prepared according to the general procedure in Example 1, Step C, substituting 2-isopropoxypyrazolo[1,5-a]pyrimidin-6-amine for 2-methoxypyrazolo[1,5-a]pyrimidin-6-amine. ¹H NMR (400 MHz, CD₃OD) δ 9.58 (s, 1H), 8.48 (s, 1H), 7.69 (m, 1H), 7.15 (m, 1H), 6.54 (s, 1H), 3.14 (m, 3H), 1.86 (m, 2H), 1.37 (d, J=7.2 Hz, 6H), 1.06 (t, J=7.4 Hz, 3H); m/z (APCI-nega) M−1=452.2.

### Example 3

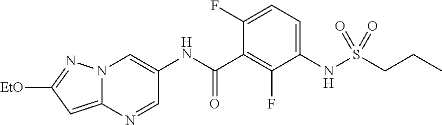

N-(2-ethoxypyrazolo[1,5-a]pyrimidin-6-yl)-2,6-difluoro-3-(propylsulfonamido)benzamide

Step A: A solution of malononitrile (10.0 g, 151 mmol), ethanol (6.97 g, 151 mmol) and ether (120 mL) was cooled to 0° C., and 2.0M HCl in ether (98.4 mL, 197 mmol) was added rapidly via an addition funnel. The reaction mixture was stirred at room temperature for 16 hours. The solids were collected by filtration and washed with ether (100 mL) to give ethyl 2-cyanoacetimidate hydrochloride (12.6 g, 56%).

Step B: A solution of ethyl 2-cyanoacetimidate hydrochloride (12.6 g, 84.8 mmol) and hydrazine (3.67 g, 114 mmol) in EtOH (50 mL) was refluxed for 16 hours. The reaction mixture was concentrated, and the residue was taken up in water (100 mL) and ethyl acetate (500 mL) and placed in an ice bath. A solution of 2N NaOH (about 6 mL) was added until the pH was adjusted to about 7. The solids were removed by filtration (discarded), and the filtrate was transferred to a separation funnel. The layers were separated, and the aqueous layer was extracted with ethyl acetate (200 mL) The combined organics were dried, filtered and concentrated. The crude product was purified by flash chromatography, eluting with hexanes/ethyl acetate (1:1), hexanes/ethyl acetate (1:2) to give 3-ethoxy-1H-pyrazol-5-amine (1.15 g, 9.04 mmol, 11% yield) as a solid. m/z (APCI-pos) M+1=128.1.

Step C: 2-Ethoxy-6-nitropyrazolo[1,5-a]pyrimidine (1.48 g, 86% yield) was prepared according to the general procedure in Example 1, Step A, substituting 3-ethoxy-1H-pyrazol-5-amine for 3-methoxy-1H-pyrazol-5-amine. m/z (APCI-nega) M−1=207.0.

Step D: 2-Ethoxypyrazolo[1,5-a]pyrimidin-6-amine (0.58 g, 45% yield) was prepared according to the general procedure in Example 1, Step B, substituting 2-ethoxy-6-nitropyrazolo[1,5-a]pyrimidine for 2-methoxy-6-nitropyrazolo[1,5-a]pyrimidine.

Step E: N-(2-Ethoxypyrazolo[1,5-a]pyrimidin-6-yl)-2,6-difluoro-3-(propylsulfonamido)benzamide (0.155 g, 22% yield) was prepared according to the general procedure in Example 1, Step C, substituting 2-ethoxypyrazolo[1,5-a]pyrimidin-6-amine for 2-methoxypyrazolo[1,5-a]pyrimidin-6-amine. ¹H NMR (400 MHz, CD₃OD) δ 9.41 (s, 1H), 8.42 (s, 1H), 7.66 (m, 1H), 7.13 (m, 1H), 6.04 (s, 1H), 4.32 (m, 2H), 3.10 (m, 2H), 1.86 (m, 2H), 1.42 (m, 3H), 1.06 (t, J=7.4 Hz, 3H); m/z (APCI-nega) M−1=438.1.

### Example 4

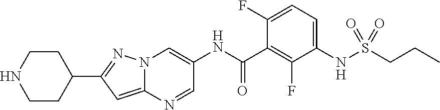

### 2,6-difluoro-N-(2-(piperidin-4-yl)pyrazolo[1,5-a]pyrimidin-6-yl)-3-(propylsulfonamido)benzamide

Step A: tert-Butyl 4-(6-nitropyrazolo[1,5-a]pyrimidin-2-yl)piperidine-1-carboxylate (0.35 g, 99% yield) was prepared according to the general procedure in Example 1, Step A, substituting tert-butyl 4-(5-amino-1H-pyrazol-3-yl)piperidine-1-carboxylate for 3-methoxy-1H-pyrazol-5-amine. m/z (APCI-nega) M−1=347.0.

Step B: tert-Butyl 4-(6-aminopyrazolo[1,5-a]pyrimidin-2-yl)piperidine-1-carboxylate (0.093 g, 97% yield) was prepared according to the general procedure in Example 1, Step B, substituting tert-butyl 4-(6-nitropyrazolo[1,5-a]pyrimidin-2-yl)piperidine-1-carboxylate for 2-methoxy-6-nitropyrazolo[1,5-a]pyrimidine. m/z (APCI-pos) M+1=317.8.

Step C: tert-Butyl 4-(6-(2,6-difluoro-3-(propylsulfonamido)benzamido)pyrazolo[1,5-a]pyrimidin-2-yl)piperidine-1-carboxylate (0.091 g, 54% yield) was prepared according to the general procedure in Example 1, Step C, substituting tert-butyl 4-(6-aminopyrazolo[1,5-a]pyrimidin-2-yl)piperidine-1-carboxylate for 2-methoxypyrazolo[1,5-a]pyrimidin-6-amine. m/z (APCI-pos) M+1=579.8.

Step D: TFA (2 mL) was added slowly to a solution of tert-butyl 4-(6-(2,6-difluoro-3-(propylsulfonamido)benzamido)pyrazolo[1,5-a]pyrimidin-2-yl)piperidine-1-carboxylate (0.091 g, 0.16 mmol) in DCM (2.0 mL). After stirring for 1 hour at room temperature, the reaction mixture was concentrated, and the residue was taken up in ethyl acetate (100 mL) and water (20 mL). The pH was adjusted to about 7 with saturated NaHCO₃, and the aqueous layer was extracted with ethyl acetate (50 mL×3). The combined organics were dried, filtered and concentrated to give 2,6-difluoro-N-(2-(piperidin-4-yl)pyrazolo[1,5-a]pyrimidin-6-yl)-3-(propylsulfonamido)benzamide as a solid (0.068 g, 0.145 mmol, 90% yield). ¹H NMR (400 MHz, CD₃OD) δ 9.64 (s, 1H), 8.50 (s, 1H), 7.68 (m, 1H), 7.16 (m, 1H), 6.62 (s, 1H), 3.51 (m, 2H), 3.22 (m, 3H), 3.12 (m, 2H), 2.35 (m, 2H), 2.09 (m, 2H), 1.87 (m, 2H), 1.06 (t, J=7.4 Hz, 3H); m/z (APCI-pos) M+1=479.2.

### Example 5

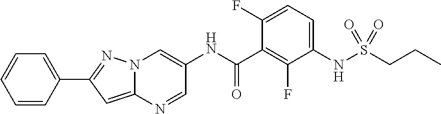

### 2,6-difluoro-N-(2-phenylpyrazolo[1,5-a]pyrimidin-6-yl)-3-(propylsulfonamido)benzamide

Step A: A suspension of 3-phenyl-1H-pyrazol-5-amine (3.08 g, 19.3 mmol) and sodium nitromalonaldehyde monohydrate (3.19 g, 20.3 mmol) in acetic acid (25 mL) was stirred at 50° C. for 1 hour. The reaction mixture was diluted with water (200 mL), and the resulting solids (4.5 g, 18.7 mmol, 97% yield) were collect by filtration and dried under vacuo to provide 6-nitro-2-phenylpyrazolo[1,5-a]pyrimidine m/z (APCI-nega) M−1=239.9.

Step B: 2-Phenylpyrazolo[1,5-a]pyrimidin-6-amine (4.0 g, 98% yield) was prepared according to the general procedure in Example 1, Step B, substituting 6-nitro-2-phenylpyrazolo[1,5-a]pyrimidine for 2-methoxy-6-nitropyrazolo[1,5-a]pyrimidine. m/z (APCI-pos) M+1=211.2.

Step C: 2,6-Difluoro-N-(2-phenylpyrazolo[1,5-a]pyrimidin-6-yl)-3-(propylsulfonamido)benzamide (0.300 g, 45% yield) was prepared according to the general procedure in Example 1, Step C, substituting 2-phenylpyrazolo[1,5-a]pyrimidin-6-amine for 2-methoxypyrazolo[1,5-a]pyrimidin-6-amine. ¹H NMR (400 MHz, (CD₃)₂SO) δ 11.39 (br s, 1H), 9.84 (br s, 1H), 9.59 (s, 1H), 8.59 (s, 1H), 8.05 (m, 2H), 7.63-7.28 (m, 6H), 3.12 (m, 2H), 1.78 (m, 2H), 1.00 (t, J=7.4 Hz, 3H); m/z (APCI-nega) M−1=470.0.

### Example 6

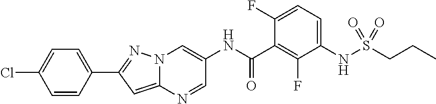

N-(2-(4-chlorophenyl)pyrazolo[1,5-a]pyrimidin-6-yl)-2,6-difluoro-3-(propylsulfonamido)benzamide

Step A: 2-(4-chlorophenyl)-6-nitropyrazolo[1,5-a]pyrimidine (0.174 g, 84% yield) was prepared according to the general procedure in Example 5, Step A, substituting 3-(4-chlorophenyl)-1H-pyrazol-5-amine for 3-phenyl-1H-pyrazol-5-amine. m/z (APCI-nega) M−1=273.9, 275.9.

Step B: 10% wt Pt/C (0.094 g, 0.048 mmol) was added to a solution of 2-(4-chlorophenyl)-6-nitropyrazolo[1,5-a]pyrimidine (0.132 g, 0.48 mmol) in a mixture of ethyl acetate/MeOH (1:1, 20 mL). The reaction mixture was purged with N₂ for 10 minutes and then placed under a balloon of H₂ for 1 hour. The Pt/C was removed by filtration, and the filtrate was concentrated. The crude product was purified by flash chromatography, eluting with hexanes/ethyl acetate (1:1) to give 2-(4-chlorophenyl)pyrazolo[1,5-a]pyrimidin-6-amine (0.084 g, 0.34 mmol, 71% yield) as a solid. m/z (APCI-pos) M+1=245.1, 247.1.

Step C: N-(2-(4-Chlorophenyl)pyrazolo[1,5-a]pyrimidin-6-yl)-2,6-difluoro-3-(propylsulfonamido)benzamide (0.095 g, 56% yield) was prepared according to the general procedure in Example 1, Step C, substituting 2-(4-chlorophenyl)pyrazolo[1,5-a]pyrimidin-6-amine for 2-methoxypyrazolo[1,5-a]pyrimidin-6-amine. ¹H NMR (400 MHz, (CD₃)₂SO) δ 11.39 (br s, 1H), 9.84 (br s, 1H), 9.59 (s, 1H), 8.59 (s, 1H), 8.07 (d, J=7.6 Hz, 2H), 7.62-7.55 (m, 3H), 7.33-7.29 (m, 2H), 3.12 (m, 2H), 1.78 (m, 2H), 1.00 (t, J=7.4 Hz, 3H); m/z (APCI-nega) M−1=504.0, 506.1.

### Example 7

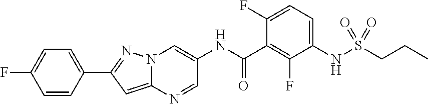

### 2,6-difluoro-N-(2-(4-fluorophenyl)pyrazolo[1,5-a]pyrimidin-6-yl)-3-(propylsulfonamido)benzamide

Step A: 2-(4-Fluorophenyl)-6-nitropyrazolo[1,5-a]pyrimidine (0.215 g, 93% yield) was prepared according to the general procedure in Example 5, Step A, substituting 3-(4-fluorophenyl)-1H-pyrazol-5-amine for 3-phenyl-1H-pyrazol-5-amine. m/z (APCI-nega) M−1=258.0.

Step B: 2-(4-Fluorophenyl)pyrazolo[1,5-a]pyrimidin-6-amine (0.160 g, 84% yield) was prepared according to the general procedure in Example 1, Step B, substituting 2-(4-fluorophenyl)-6-nitropyrazolo[1,5-a]pyrimidine for 2-methoxy-6-nitropyrazolo[1,5-a]pyrimidine. m/z (APCI-pos) M+1=229.2.

Step C: 2,6-Difluoro-N-(2-(4-fluorophenyl)pyrazolo[1,5-a]pyrimidin-6-yl)-3-(propylsulfonamido)benzamide (0.110 g, 43% yield) was prepared according to the general procedure in Example 1, Step C, substituting 2-(4-fluorophenyl)pyrazolo[1,5-a]pyrimidin-6-amine for 2-methoxypyrazolo[1,5-a]pyrimidin-6-amine. ¹H NMR (400 MHz, (CD₃)₂SO) δ 11.37 (br s, 1H), 9.81 (br s, 1H), 9.57 (s, 1H), 8.56 (s, 1H), 8.07 (m, 2H), 7.57 (m, 1H), 7.33-7.26 (m, 4H), 3.12 (m, 2H), 1.78 (m, 2H), 1.00 (t, J=7.4 Hz, 3H); m/z (APCI-nega) M−1=488.1.

### Example 8

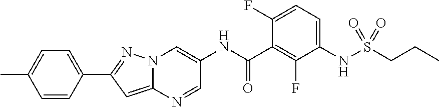

### 2,6-difluoro-3-(propylsulfonamido)-N-(2-p-tolylpyrazolo[1,5-a]pyrimidin-6-yl)benzamide

Step A: 6-Nitro-2-p-tolylpyrazolo[1,5-a]pyrimidine (0.215 g, 93% yield) was prepared according to the general procedure in Example 5, Step A, substituting 3-p-tolyl-1H-pyrazol-5-amine for 3-phenyl-1H-pyrazol-5-amine. m/z (APCI-nega) M−1=254.0.

Step B: 2-p-Tolylpyrazolo[1,5-a]pyrimidin-6-amine (0.185 g, 98% yield) was prepared according to the general procedure in Example 1, Step B, substituting 6-nitro-2-p-tolylpyrazolo[1,5-a]pyrimidine for 2-methoxy-6-nitropyrazolo[1,5-a]pyrimidine. m/z (APCI-pos) M+1=225.1.

Step C: 2,6-Difluoro-3-(propylsulfonamido)-N-(2-p-tolylpyrazolo[1,5-a]pyrimidin-6-yl)benzamide (0.095 g, 39% yield) was prepared according to the general procedure in Example 1, Step C, substituting 2-p-tolylpyrazolo[1,5-a]pyrimidin-6-amine for 2-methoxypyrazolo[1,5-a]pyrimidin-6-amine. ¹H NMR (400 MHz, (CD₃)₂SO) δ 11.34 (br s, 1H), 9.81 (br s, 1H), 9.54 (s, 1H), 8.54 (s, 1H), 7.90 (d, J=7.6 Hz, 2H), 7.85 (m, 1H), 7.28 (m, 3H), 7.21 (s, 1H), 3.12 (m, 2H), 2.34 (s, 3H), 1.78 (m, 2H), 1.00 (t, J=7.4 Hz, 3H); m/z (APCI-nega) M−1=484.1.

### Example 9

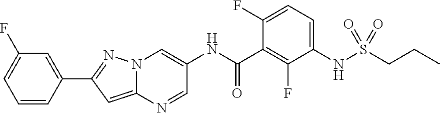

### 2,6-difluoro-N-(2-(3-fluorophenyl)pyrazolo[1,5-a]pyrimidin-6-yl)-3-(propylsulfonamido)benzamide

Step A: 2-(3-Fluorophenyl)-6-nitropyrazolo[1,5-a]pyrimidine (0.214 g, 93% yield) was prepared according to the general procedure in Example 5, Step A, substituting 3-(3-fluorophenyl)-1H-pyrazol-5-amine for 3-phenyl-1H-pyrazol-5-amine. m/z (APCI-nega) M−1=257.9.

Step B: 2-(3-Fluorophenyl)pyrazolo[1,5-a]pyrimidin-6-amine (0.160 g, 84% yield) was prepared according to the general procedure in Example 1, Step B, substituting 2-(3-fluorophenyl)-6-nitropyrazolo[1,5-a]pyrimidine for 2-methoxy-6-nitropyrazolo[1,5-a]pyrimidine. m/z (APCI-pos) M+1=229.1.

Step C: 2,6-Difluoro-N-(2-(3-fluorophenyl)pyrazolo[1,5-a]pyrimidin-6-yl)-3-(propylsulfonamido)benzamide (0.080 g, 44% yield) was prepared according to the general procedure in Example 1, Step C, substituting 2-(3-fluorophenyl)pyrazolo[1,5-a]pyrimidin-6-amine for 2-methoxypyrazolo[1,5-a]pyrimidin-6-amine. ¹H NMR (400 MHz, (CD₃)₂SO) 11.39 (br s, 1H), 9.81 (br s, 1H), 9.58 (s, 1H), 8.58 (s, 1H), 7.85 (m, 2H), 7.61-7.49 (m, 2H), 7.35 (s, 1H), 7.31-7.21 (m, 2H), 3.12 (m, 2H), 1.78 (m, 2H), 1.00 (t, J=7.4 Hz, 3H); m/z (APCI-nega) M−1=488.0.

### Example 10

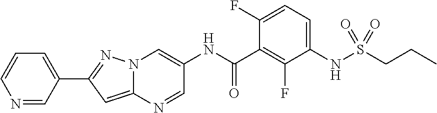

### 2,6-difluoro-3-(propylsulfonamido)-N-(2-(pyridin-3-yl)pyrazolo[1,5-a]pyrimidin-6-yl)benzamide

Step A: 6-Nitro-2-(pyridin-3-yl)pyrazolo[1,5-a]pyrimidine (0.220 g, 70% yield) was prepared according to the general procedure in Example 5, Step A, substituting 3-(pyridin-3-yl)-1H-pyrazol-5-amine dihydrochloride for 3-phenyl-1H-pyrazol-5-amine. m/z (APCI-nega) M−1=241.0.

Step B: 2-(Pyridin-3-yl)pyrazolo[1,5-a]pyrimidin-6-amine (0.023 g, 12% yield) was prepared according to the general procedure in Example 1, Step B, substituting 6-nitro-2-(pyridin-3-yl)pyrazolo[1,5-a]pyrimidine for 2-methoxy-6-nitropyrazolo[1,5-a]pyrimidine. m/z (APCI-pos) M+1=212.3.

Step C: 2,6-Difluoro-3-(propylsulfonamido)-N-(2-(pyridin-3-yl)pyrazolo[1,5-a]pyrimidin-6-yl)benzamide (0.030 g, 58% yield) was prepared according to the general procedure in Example 1, Step C, substituting 2-(pyridin-3-yl)pyrazolo[1,5-a]pyrimidin-6-amine for 2-methoxypyrazolo[1,5-a]pyrimidin-6-amine. ¹H NMR (400 MHz, (CD₃)₂SO) δ 11.39 (br s, 1H), 9.81 (br s, 1H), 9.60 (s, 1H), 9.23 (s, 1H), 8.59 (m, 2H), 8.37 (d, J=8.8 Hz, 1H), 7.61-7.45 (m, 2H), 7.40 (s, 1H), 7.28 (m, 1H), 3.12 (m, 2H), 1.78 (m, 2H), 1.00 (t, J=7.4 Hz, 3H); m/z (APCI-pos) M+1=473.1.

### Example 11

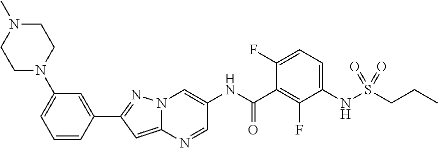

### 2,6-difluoro-N-(2-(3-(4-methylpiperazin-1-yl)phenyl)pyrazolo[1,5-a]pyrimidin-6-yl)-3-(propylsulfonamido)benzamide

Step A: Potassium 2-methylbutan-2-olate (1.23 g, 2.44 mmol, 25% wt in toluene) was added dropwise to a solution of acetonitrile (0.100 g, 2.44 mmol) in anhydrous THF (5.0 mL), followed by methyl 3-(4-methylpiperazin-1-yl)benzoate (0.856 g, 3.65 mmol). The reaction mixture was stirred at room temperature for 1 hour before quenching with water (10.0 mL). The pH was adjusted to about 7 with CH₃COOH, and the aqueous layer was extracted with ethyl acetate (100 mL×3). The combined organics were dried, filtered and concentrated. The crude product was purified by flash column chromatography, eluting with MeOH/DCM (40:1) to give 3-(3-(4-methylpiperazin-1-yl)phenyl)-3-oxopropanenitrile (0.141 g, 0.58 mmol, 24% yield) as a solid. m/z (APCI-pos) M+1=244.2.

Step B: A solution of 3-(3-(4-methylpiperazin-1-yl)phenyl)-3-oxopropanenitrile (0.141 g, 0.58 mmol) and hydrazine (0.056 g, 1.74 mmol) in EtOH (10 mL) was refluxed for 16 hours. The reaction mixture was then cooled to room temperature and concentrated. The crude product was purified by flash column chromatography, eluting with DCM/MeOH (20:1), DCM/MeOH (10:1) to give 3-(3-(4-methylpiperazin-1-yl)phenyl)-1H-pyrazol-5-amine (0.085 g, 0.33 mmol, 57% yield) as a solid. m/z (APCI-pos) M+1=258.1.

Step C: 2-(3-(4-Methylpiperazin-1-yl)phenyl)-6-nitropyrazolo[1,5-a]pyrimidine (0.110 g, 99% yield) was prepared according to the general procedure in Example 5, Step A, substituting 3-(3-(4-methylpiperazin-1-yl)phenyl)-1H-pyrazol-5-amine for 3-phenyl-1H-pyrazol-5-amine. m/z (APCI-pos) M+1=339.2.

Step D: 2-(3-(4-Methylpiperazin-1-yl)phenyl)pyrazolo[1,5-a]pyrimidin-6-amine (0.100 g, 98% yield) was prepared according to the general procedure in Example 1, Step B, substituting 2-(3-(4-methylpiperazin-1-yl)phenyl)-6-nitropyrazolo[1,5-a]pyrimidine for 2-methoxy-6-nitropyrazolo[1,5-a]pyrimidine. m/z (APCI-pos) M+1=309.3.

Step E: 2,6-Difluoro-N-(2-(3-(4-methylpiperazin-1-yl)phenyl)pyrazolo[1,5-a]pyrimidin-6-yl)-3-(propylsulfonamido)benzamide (0.053 g, 28% yield) was prepared according to the general procedure in Example 1, Step C, substituting 2-(3-(4-methylpiperazin-1-yl)phenyl)pyrazolo[1,5-a]pyrimidin-6-amine for 2-methoxypyrazolo[1,5-a]pyrimidin-6-amine. ¹H NMR (400 MHz, CDCl₃) δ 9.58 (s, 1H), 8.36 (s, 1H), 8.23 (s, 1H), 7.68 (m, 1H), 7.57 (s, 1H), 7.43-7.32 (m, 2H), 7.06-6.94 (m, 3H), 3.31 (m, 4H), 3.10 (m, 2H), 2.62 (m, 4H), 2.38 (s, 3H), 1.88 (m, 2H), 1.06 (t, J=7.4 Hz, 3H); m/z (APCI-pos) M+1=570.3.

### Example 12

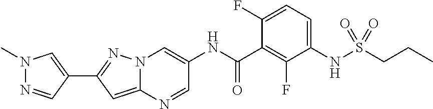

### 2,6-difluoro-N-(2-(1-methyl-1H-pyrazol-4-yl)pyrazolo[1,5-a]pyrimidin-6-yl)-3-(propylsulfonamido)benzamide

Step A: (Trimethylsilyl)diazomethane (12.0 mL, 24 mmol, 2.0M solution in hexanes) was added dropwise via an addition funnel to a cold (0° C.) solution of 1-methyl-1H-pyrazole-4-carboxylic acid (0.59 g, 4.7 mmol) in MeOH (20 mL). The reaction mixture was stirred for 20 minutes and then concentrated. The crude product was partitioned between ethyl acetate (100 mL) and water (50 mL). The organics were dried, filtered and concentrated. The crude product was purified by flash chromatography, eluting with hexanes/ethyl acetate (4:1) to give methyl 1-methyl-1H-pyrazole-4-carboxylate (0.50 g, 3.6 mmol, 76% yield) as a solid. m/z (APCI-pos) M+1=141.1.

Step B: 3-(1-Methyl-1H-pyrazol-4-yl)-3-oxopropanenitrile (0.075 g, 17% yield) was prepared according to the general procedure in Example 11, Step A, substituting methyl 1-methyl-1H-pyrazole-4-carboxylate for methyl 3-(4-methylpiperazin-1-yl)benzoate.

Step C: 1′-methyl-1H,1′H-3,4′-bipyrazol-5-amine (0.031 g, 38% yield) was prepared according to the general procedure in Example 11, Step B, substituting 3-(1-methyl-1H-pyrazol-4-yl)-3-oxopropanenitrile for 3-(3-(4-methylpiperazin-1-yl)phenyl)-3-oxopropanenitrile. m/z (APCI-pos) M+1=164.2.

Step D: 2-(1-Methyl-1H-pyrazol-4-yl)-6-nitropyrazolo pyrimidine (0.045 g, 98% yield) was prepared according to the general procedure in Example 5, Step A, substituting 1′-methyl-1H,1′H-3,4′-bipyrazol-5-amine for 3-phenyl-1H-pyrazol-5-amine. m/z (APCI-pos) M+1=245.1.

Step E: 2-(1-Methyl-1H-pyrazol-4-yl)pyrazolo[1,5-a]pyrimidin-6-amine (0.018 g, 42% yield) was prepared according to the general procedure in Example 1, Step B, substituting 2-(1-methyl-1H-pyrazol-4-yl)-6-nitropyrazolo[1,5-a]pyrimidine for 2-methoxy-6-nitropyrazolo[1,5-a]pyrimidine. m/z (APCI-pos) M+1=215.1.

Step F: 2,6-Difluoro-N-(2-(1-methyl-1H-pyrazol-4-yl)pyrazolo[1,5-a]pyrimidin-6-yl)-3-(propylsulfonamido)benzamide (0.053 g, 28% yield) was prepared according to the general procedure in Example 1, Step C, substituting 2-(1-methyl-1H-pyrazol-4-yl)pyrazolo[1,5-a]pyrimidin-6-amine for 2-methoxypyrazolo[1,5-a]pyrimidin-6-amine. ¹H NMR (400 MHz, CD₃OD) δ 9.63 (s, 1H), 8.49 (s, 1H), 8.12 (s, 1H), 7.96 (s, 1H), 7.67 (m, 1H), 7.16 (m, 1H), 6.84 (s, 1H), 3.96 (s, 3H), 3.12 (m, 2H), 1.88 (m, 2H), 1.06 (t, J=7.4 Hz, 3H); m/z (APCI-pos) M+1=476.2.

### Example 13

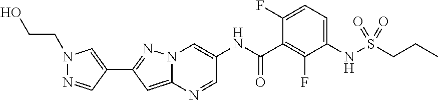

### 2,6-difluoro-N-(2-(1-(2-hydroxyethyl)-1H-pyrazol-4-yl)pyrazolo[1,5-a]pyrimidin-6-yl)-3-(propylsulfonamido)benzamide

Step A: A solution of 2-hydrazinylethanol (2.76 g, 32.7 mmol) in EtOH (10 mL) was added dropwise to a solution of ethyl 2-formyl-3-oxopropanoate (4.71 g, 32.7 mmol, prepared as described in Bertz, Steven H., et al. “New preparations of ethyl 3,3-diethoxypropionate and ethoxycarbonylmalondialdehyde. Copper(I) catalyzed acetal formation from a conjugated triple bond.” J. Org. Chem. Vol. 47 (1982): pp. 2216-2217) in EtOH (20 mL) at 0° C. The reaction mixture was stirred at room temperature for 48 hours and then concentrated to give ethyl 1-(2-hydroxyethyl)-1H-pyrazole-4-carboxylate (5.8 g, 31.5 mmol, 96% yield) as an oil. m/z (APCI-pos) M+1=185.1.

Step B: 3-(1-(2-Hydroxyethyl)-1H-pyrazol-4-yl)-3-oxopropanenitrile (0.120 g, 28% yield) was prepared according to the general procedure in Example 11, Step A, substituting ethyl 1-(2-hydroxyethyl)-1H-pyrazole-4-carboxylate for methyl 3-(4-methylpiperazin-1-yl)benzoate.

Step C: 2-(5-Amino-1H,1′H-3,4′-bipyrazol-1′-yl)ethanol (0.119 g, 92% yield) was prepared according to the general procedure in Example 11, Step B, substituting 3-(1-(2-hydroxyethyl)-1H-pyrazol-4-yl)-3-oxopropanenitrile for 3-(3-(4-methylpiperazin-1-yl)phenyl)-3-oxopropanenitrile. m/z (APCI-pos) M+1=194.1.

Step D: 2-(4-(6-Nitropyrazolo[1,5-a]pyrimidin-2-yl)-1H-pyrazol-1-yl)ethanol (0.137 g, 81% yield) was prepared according to the general procedure in Example 5, Step A, substituting 2-(5-amino-1H,1′H-3,4′-bipyrazol-1′-yl)ethanol for 3-phenyl-1H-pyrazol-5-amine. m/z (APCI-pos) M+1=275.1.

Step E: 2-(4-(6-Aminopyrazolo[1,5-a]pyrimidin-2-yl)-1H-pyrazol-1-yl)ethanol (0.090 g, 75% yield) was prepared according to the general procedure in Example 1, Step B, substituting 2-(4-(6-nitropyrazolo[1,5-a]pyrimidin-2-yl)-1H-pyrazol-1-yl)ethanol for 2-methoxy-6-nitropyrazolo[1,5-a]pyrimidine. m/z (APCI-pos) M+1=245.1.

Step F: 2,6-Difluoro-N-(2-(1-(2-hydroxyethyl)-1H-pyrazol-4-yl)pyrazolo[1,5-a]pyrimidin-6-yl)-3-(propylsulfonamido)benzamide (0.102 g, 55% yield) was prepared according to the general procedure in Example 1, Step C, substituting 2-(4-(6-aminopyrazolo[1,5-a]pyrimidin-2-yl)-1H-pyrazol-1-yl)ethanol for 2-methoxypyrazolo[1,5-a]pyrimidin-6-amine. ¹H NMR (400 MHz, CD₃OD) δ 9.63 (s, 1H), 8.49 (s, 1H), 8.19 (s, 1H), 8.01 (s, 1H), 7.67 (m, 1H), 7.16 (m, 1H), 6.86 (s, 1H), 4.30 (m, 2H), 3.94 (m, 2H), 3.12 (m, 2H), 1.88 (m, 2H), 1.06 (t, J=7.4 Hz, 3H); m/z (APCI-pos) M+1=506.2.

### Example 14

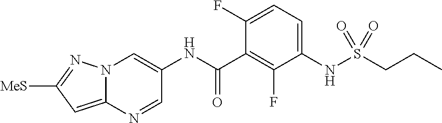

### 2,6-difluoro-N-(2-(methylthio)pyrazolo[1,5-a]pyrimidin-6-yl)-3-(propylsulfonamido)benzamide

Step A: Ethyl 2-cyanoacetate (2.00 mL, 18.7 mmol) was added dropwise to a cold suspension (0° C.) of NaH (1.50 g, 37.5 mmol, 60% in mineral oil) in benzene (20 mL), followed by CS₂ (1.7 mL, 28.1 mmol). DMF (4 mL) was added slowly, and the mixture was stirred for 30 minutes before MeI (3.52 mL, 56.2 mmol) was added. The resulting mixture was stirred at room temperature overnight. Benzene (50 mL) was added, and the yellow slurry was quenched with ice-water. The organic layer was separated, dried, filtered and concentrated. The crude product was purified by column chromatography, eluting with hexanes/ethyl acetate (4:1) to give 2-cyano-3,3-bis(methylthio)acrylate (2.2 g, 54%) as a solid.

Step B: A solution of ethyl 2-cyano-3,3-bis(methylthio)acrylate (2.2 g, 10.1 mmol) and hydrazine (0.325 mL, 10.1 mmol) in 2-propanol (20 mL) was heated at reflux overnight. The reaction mixture was cooled to room temperature and concentrated. The crude product was purified by column chromatography, eluting with hexanes/ethyl acetate (1:1) to give ethyl 5-amino-3-(methylthio)-1H-pyrazole-4-carboxylate (1.2 g, 59%) as a solid. m/z (APCI-pos) M+1=202.0.

Step C: Ethyl 5-amino-3-(methylthio)-1H-pyrazole-4-carboxylate (1.2 g, 5.96 mmol) was dissolved in a solution of LiOH (1.14 g, 47.7 mmol) in MeOH/H₂O (9:1, 40 mL). The resulting solution was heated at reflux for 72 hours. The reaction mixture was cooled to room temperature and concentrated. The residue was diluted with water, and the insoluble material was removed by filtration. The filtrate was extracted with ethyl acetate (100 mL×4), and the combined organics were dried, filtered and concentrated to give 3-(methylthio)-1H-pyrazol-5-amine (0.61 g, 79%) as a solid. m/z (APCI-pos) M+1=130.0.

Step D: 2-(Methylthio)-6-nitropyrazolo[1,5-a]pyrimidine (0.189 g, 89% yield) was prepared according to the general procedure in Example 1, Step A, substituting 3-(methylthio)-1H-pyrazol-5-amine for 3-methoxy-1H-pyrazol-5-amine. m/z (APCI-nega) M−1=209.8.

Step E: 2-(Methylthio)pyrazolo[1,5-a]pyrimidin-6-amine (0.150 g, 94% yield) was prepared according to the general procedure in Example 1, Step B, substituting 2-(methylthio)-6-nitropyrazolo[1,5-a]pyrimidine for 2-methoxy-6-nitropyrazolo[1,5-a]pyrimidine. m/z (APCI-pos) M+1=181.0.

Step F: 2,6-Difluoro-N-(2-(methylthio)pyrazolo[1,5-a]pyrimidin-6-yl)-3-(propylsulfonamido)benzamide (0.010 g, 3% yield) was prepared according to the general procedure in Example 1, Step C, substituting 2-(methylthio)pyrazolo[1,5-a]pyrimidin-6-amine for 2-methoxypyrazolo[1,5-a]pyrimidin-6-amine. ¹H NMR (400 MHz, CD₃OD) δ 9.56 (s, 1H), 8.49 (s, 1H), 7.67 (m, 1H), 7.16 (m, 1H), 6.58 (s, 1H), 3.12 (m, 2H), 2.62 (s, 3H), 1.88 (m, 2H), 1.06 (t, J=7.4 Hz, 3H); m/z (APCI-nega) M−1=440.0.

### Example 15

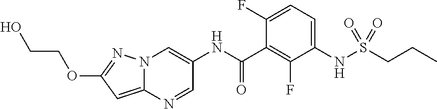

### 2,6-difluoro-N-(2-(2-hydroxyethoxy)pyrazolo[1,5-a]pyrimidin-6-yl)-3-(propylsulfonamido)benzamide

Step A: M NaOH (46.5 mL, 46.5 mmol) was added to a solution of ethyl 5-amino-3-(2-hydroxyethoxy)-1H-pyrazole-4-carboxylate (2.00 g, 9.29 mmol, prepared as described in Neidlein, Richard, et al. “Heterocyclic Compounds from 2-(Alkoxycarbonylcyanomethylene)-1,3-dioxolanes.” J. Het. Chem. Vol. 26 (1989): pp. 1335-1340) in ethanol (30 mL), and the mixture was refluxed overnight. The solution was washed with DCM with 25% isopropyl alcohol (“IPA”) and then acidified to a pH of 3 with concentrated HCl. Gas evolution was observed. The solution was washed with DCM with 25% IPA, and the aqueous layer was evaporated. The residue was treated with methanol, filtered, and the filtrate was evaporated to yield crude 2-(5-amino-1H-pyrazol-3-yloxy)ethanol (3.28 g) along with inorganic salts. m/z (APCI-pos) M+1=144.0.

Step B: 2-(6-Nitropyrazolo[1,5-a]pyrimidin-2-yloxy)ethanol (0.41 g, 52% yield) was prepared according to the general procedure in Example 1, Step A, substituting 2-(5-amino-1H-pyrazol-3-yloxy)ethanol for 3-methoxy-1H-pyrazol-5-amine.

Step C: 2-(6-Aminopyrazolo[1,5-a]pyrimidin-2-yloxy)ethanol (0.27 g, 76% yield) was prepared according to the general procedure in Example 1, Step B, substituting 2-(6-nitropyrazolo[1,5-a]pyrimidin-2-yloxy)ethanol for 2-methoxy-6-nitropyrazolo[1,5-a]pyrimidine. m/z (APCI-pos) M+1=195.1.

Step D: 2,6-Difluoro-N-(2-(2-hydroxyethoxy)pyrazolo[1,5-a]pyrimidin-6-yl)-3-(propylsulfonamido)benzamide (0.183 g, 62% yield) was prepared according to the general procedure in Example 1, Step C, substituting 2-(6-aminopyrazolo[1,5-a]pyrimidin-2-yloxy)ethanol for 2-methoxypyrazolo[1,5-a]pyrimidin-6-amine. ¹H NMR (400 MHz, CD₃OD) δ 9.44 (s, 1H), 8.45 (s, 1H), 7.67 (m, 1H), 7.16 (m, 1H), 6.09 (s, 1H), 4.37 (m, 2H), 3.91 (m, 2H), 3.12 (m, 2H), 1.88 (m, 2H), 1.06 (t, J=7.4 Hz, 3H); m/z (APCI-nega) M−1=454.1.

### Example 16

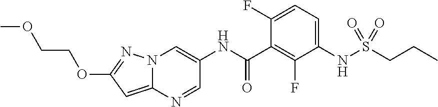

### 2,6-difluoro-N-(2-(2-methoxyethoxy)pyrazolo[1,5-a]pyrimidin-6-yl)-3-(propylsulfonamido)benzamide

Step A: Diisopropyl azodicarboxylate (5.05 g, 23.7 mmol) was added dropwise over a period of 15 minutes (internal temp <15° C.) to a cold (0° C.) solution of 5-amino-1H-pyrazol-3-ol (2.0 g, 19.8 mmol) and PPh₃ (6.23 g, 23.7 mmol) in DCM (30 mL). After stirring at 0° C. for 1 hour, 2-methoxyethanol (1.81 g, 23.7 mmol) was added dropwise over 10 minutes. The reaction mixture was allowed to warm up to room temperature over 1 hour and stirred under N₂ for 3 days. The solids were removed by filtration, and the filter cake was washed with DCM. The DCM later was extracted with 1N HCl (2×50 mL). The combined aqueous layer was washed with DCM (100 mL), and the DCM layer was discarded. The aqueous layer was basified to about pH 8 with 2N NaOH and extracted with ethyl acetate (200 mL×3). The combined organics were dried, filtered and concentrated. The crude product was purified on by flash chromatography, eluting with ethyl acetate/MeOH (50:1) to give 3-(2-methoxyethoxy)-1H-pyrazol-5-amine (0.40 g, 2.55 mmol, 13% yield) as an oil. m/z (APCI-pos) M+1=158.2.

Step B: 2-(2-Methoxyethoxy)-6-nitropyrazolo[1,5-a]pyrimidine (0.38 g, 63% yield) was prepared according to the general procedure in Example 1, Step A, substituting 3-(2-methoxyethoxy)-1H-pyrazol-5-amine for 3-methoxy-1H-pyrazol-5-amine. m/z (APCI-nega) M−1=237.0.

Step C: 2-(2-Methoxyethoxy)pyrazolo[1,5-a]pyrimidin-6-amine (0.16 g, 48% yield) was prepared according to the general procedure in Example 1, Step B, substituting 2-(2-methoxyethoxy)-6-nitropyrazolo[1,5-a]pyrimidine for 2-methoxy-6-nitropyrazolo[1,5-a]pyrimidine. m/z (APCI-pos) M+1=209.0.

Step D: 2,6-Difluoro-N-(2-(2-methoxyethoxy)pyrazolo[1,5-a]pyrimidin-6-yl)-3-(propylsulfonamido)benzamide (0.117 g, 61% yield) was prepared according to the general procedure in Example 1, Step C, substituting 2-(2-methoxyethoxy)pyrazolo[1,5-a]pyrimidin-6-amine for 2-methoxypyrazolo[1,5-a]pyrimidin-6-amine. ¹H NMR (400 MHz, CD₃OD) δ 9.44 (s, 1H), 8.45 (s, 1H), 7.67 (m, 1H), 7.16 (m, 1H), 6.09 (s, 1H), 4.43 (m, 2H), 3.79 (m, 2H), 3.43 (s, 3H), 3.12 (m, 2H), 1.88 (m, 2H), 1.06 (t, J=7.4 Hz, 3H); m/z (APCI-nega) M−1=468.1.

### Example 17

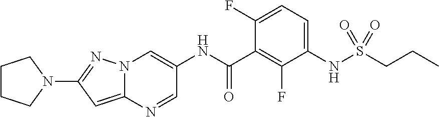

### 2,6-difluoro-3-(propylsulfonamido)-N-(2-(pyrrolidin-1-yl)pyrazolo[1,5-a]pyrimidin-6-yl)benzamide

Step A: A solution of NaOH (40 g, 999 mmol) in H₂O (40 mL) was added slowly via an addition funnel (so that the internal temperature do not exceed 10° C.) to a cold (0° C.) solution of ethyl 2-cyanoacetate (53.3 mL, 499.5 mmol) and carbon disulfide (30.2 mL, 499.5 mmol) in absolute EtOH (600 mL) Once the addition was complete, the reaction was allowed stirred at room temperature for 10 minutes and then cooled to 5° C. again. The resulting solids were isolated by filtration and washed with EtOH (100 mL), ether (500 mL) and dried in vacuo to give sodium 2-cyano-3-ethoxy-3-oxoprop-1-ene-1,1-bis(thiolate) as a solid (110.0 g, 97%).

Step B: 2-Cyano-3-ethoxy-3-oxoprop-1-ene-1,1-bis(thiolate) (110.0 g, 490 mmol) was introduced to a solution of NaOH (32.8 g, 819.4 mmol) dissolved in water (230 mL). The mixture was heated to 40° C. for 5 hours and then cooled to room temperature. The solution was diluted with EtOH (410 mL), and the layers were separated. The low layer was diluted with water to a total volume of 770 mL. The solution was cooled to 5° C. and dimethyl sulfate (77.5 mL, 819.4 mmol) was added at a rate such that the internal temperature was maintained below 15° C. Once the addition was complete, the temperature was held at 15° C. for 20 minutes and then between 28° C.-30° C. for 20 minutes. The solution was cooled to 15° C. and filtered. The filtrate was acidified with 4N HCl to about pH 2. The resulting solids were collected by filtration and dried under vacuum to give 2-cyano-3,3-bis(methylthio)acrylic acid (35.1 g, 34%) as a solid.

Step C: Pyrrolidine (0.387 g, 5.44 mmol) and triethylamine (0.275 g, 2.72 mmol) were added dropwise to a cold (0° C.) solution of 2-cyano-3,3-bis(methylthio)acrylic acid (0.515 g, 2.72 mmol) in MeOH (6 mL), and the mixture was stirred at room temperature overnight. Next, the reaction mixture was concentrated on a rotovap taking care not to heat the water bath (bath temperature about 20° C.). The crude (Z)-3-(methylthio)-3-(pyrrolidin-1-yl)acrylonitrile was used directly in Step D.

Step D: A mixture of (Z)-3-(methylthio)-3-(pyrrolidin-1-yl)acrylonitrile (0.458 g, 2.72 mmol) and hydrazine monohydrate (0.267 g, 8.17 mmol) in EtOH (6 mL) was heated to reflux for 16 hours. After cooling to room temperature, the reaction mixture was concentrated. The residue was purified by column chromatography, eluting with ethyl acetate, DCM/MeOH (9:1) to give 3-(pyrrolidin-1-yl)-1H-pyrazol-5-amine (0.240 g, 58% over 2 steps) as an oil.

Step E: 6-Nitro-2-(pyrrolidin-1-yl)pyrazolo[1,5-a]pyrimidine (0.318 g, 87% yield) was prepared according to the general procedure in Example 1, Step A, substituting 3-(pyrrolidin-1-yl)-1H-pyrazol-5-amine for 3-methoxy-1H-pyrazol-5-amine.

Step F: 2-(pyrrolidin-1-yl)pyrazolo[1,5-a]pyrimidin-6-amine (0.260 g, 94% yield) was prepared according to the general procedure in Example 1, Step B, substituting 6-nitro-2-(pyrrolidin-1-yl)pyrazolo[1,5-a]pyrimidine for 2-methoxy-6-nitropyrazolo[1,5-a]pyrimidine. m/z (APCI-pos) M+1=204.2.

Step G: 2,6-Difluoro-3-(propylsulfonamido)-N-(2-(pyrrolidin-1-yl)pyrazolo[1,5-a]pyrimidin-6-yl)benzamide (0.260 g, 86% yield) was prepared according to the general procedure in Example 1, Step C, substituting 2-(pyrrolidin-1-yl)pyrazolo[1,5-a]pyrimidin-6-amine for 2-methoxypyrazolo[1,5-a]pyrimidin-6-amine. ¹H NMR (400 MHz, CD₃OD) δ 9.33 (s, 1H), 8.31 (s, 1H), 7.66 (m, 1H), 7.14 (m, 1H), 5.81 (s, 1H), 3.41 (m, 4H), 3.12 (m, 2H), 2.04 (m, 4H), 1.88 (m, 2H), 1.06 (t, J=7.4 Hz, 3H); m/z (APCI-nega) M−1=463.1.

### Example 18

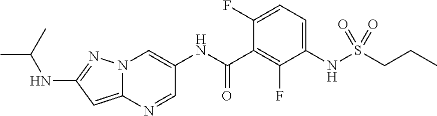

### 2,6-difluoro-N-(2-(isopropylamino)pyrazolo[1,5-a]pyrimidin-6-yl)-3-(propylsulfonamido)benzamide

Step A: (Z)-3-(Isopropylamino)-3-(methylthio)acrylonitrile was prepared according to the general procedure in Example 17, Step C, substituting isopropyl amine for pyrrolidine.

Step B: N3-Isopropyl-1H-pyrazole-3,5-diamine (0.231 g, 62% over Steps A and B) was prepared as an oil according to the general procedure in Example 17, Step D, substituting (Z)-3-(isopropylamino)-3-(methylthio)acrylonitrile for (Z)-3-(methylthio)-3-(pyrrolidin-1-yl)acrylonitrile.

Step C: N-Isopropyl-6-nitropyrazolo[1,5-a]pyrimidin-2-amine (0.060 g, 36% yield) was prepared according to the general procedure in Example 1, Step A, substituting N3-isopropyl-1H-pyrazole-3,5-diamine for 3-methoxy-1H-pyrazol-5-amine. m/z (APCI-nega) M−1=220.1.

Step D: N2-Isopropylpyrazolo[1,5-a]pyrimidine-2,6-diamine (0.050 g, 96% yield) was prepared according to the general procedure in Example 1, Step B, substituting N-isopropyl-6-nitropyrazolo[1,5-a]pyrimidin-2-amine for 2-methoxy-6-nitropyrazolo[1,5-a]pyrimidine. m/z (APCI-pos) M+1=192.1.

Step E: 2,6-Difluoro-N-(2-(isopropylamino)pyrazolo[1,5-a]pyrimidin-6-yl)-3-(propylsulfonamido)benzamide (0.117 g, 61% yield) was prepared according to the general procedure in Example 1, Step C, substituting N2-isopropylpyrazolo[1,5-a]pyrimidine-2,6-diamine for 2-methoxypyrazolo[1,5-a]pyrimidin-6-amine. m/z (APCI-nega) M−1=451.1.

The following compounds in Table 1 were prepared according to the general procedure of the Example Number given in the Method column using appropriate intermediates.

TABLE 1

| Ex. | | | | Meth- |
|---|---|---|---|---|
| # | Structure | Name | MS/NMR | od |
| 19 | 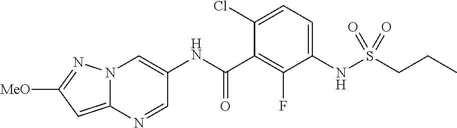 | 6-chloro-2- fluoro-N-(2- methoxypyrazolo [1,5-a]pyrimidin- 6-yl)-3- (propylsulfonamido) benzamide | ¹H NMR (400 MHz, CD₃OD) δ 9.43 (s, 1H), 8.44 (s, 1H), 7.66 (m, 1H), 7.37 (m, 1H), 6.07 (s, 1H), 4.01 (s, 3H), 3.15 (m, 2H), 1.87 (m, 2H), 1.06 (t, J = 8.0 Hz, 3H); m/z (APCI-nega) M − 1 = 440.1, 442.1 | 1 |
| 20 | 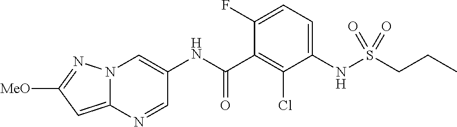 | 2-chloro-6- fluoro-N-(2- methoxypyrazolo [1,5-a]pyrimidin- 6-yl)-3- (propylsulfonamido) benzamide | ¹H NMR (400 MHz, CD₃OD) δ 9.43 (s, 1H), 8.44 (s, 1H), 7.72 (m, 1H), 7.29 (m, 1H), 6.07 (s, 1H), 4.01 (s, 3H), 3.12 (m, 2H), 1.87 (m, 2H), 1.06 (t, J = 8.0 Hz, 3H); m/z (APCI-nega) M − 1 = 440.1, 442.1 | 1 |
| 21 | 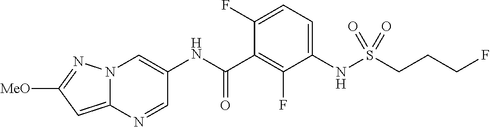 | 2,6-difluoro-3-(3- fluoropropyl- sulfonamido)-N-(2- methoxypyrazolo [1,5-a]pyrimidin- 6-yl)benzamide | ¹H NMR (400 MHz, (CD₃)₂SO) δ 11.22 (br s, 1H), 9.93 (br s, 1H), 9.33 (s, 1H), 8.48 (s, 1H), 7.56 (m, 1H), 7.27 (m, 1H), 6.17 (s, 1H), 4.58 (m, 1H), 4.47 (m, 1H), 3.92 (s, 3H), 3.23 (m, 2H), 2.10 (m, 2H); m/z (APCI-pos) M + 1 = 444.1 | 1 |
| 22 | 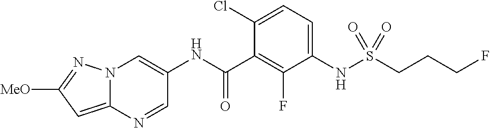 | 6-chloro-2- fluoro-3-(3- fluoropropyl- sulfonamido)-N-(2- methoxypyrazolo [1,5-a]pyrimidin- 6-yl)benzamide | ¹H NMR (400 MHz, CD₃OD) δ 9.43 (s, 1H), 8.43 (s, 1H), 7.65 (m, 1H), 7.37 (m, 1H), 6.08 (s, 1H), 4.60 (m, 1H), 4.49 (m, 1H), 4.01 (s, 3H), 3.23 (m, 2H), 2.20 (m, 2H); m/z (APCI- pos) M + 1 = 460.1, 462.1 | 1 |
| 23 | 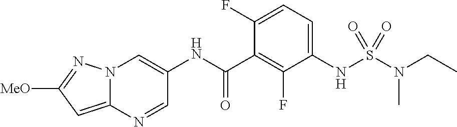 | 3-(N-ethyl-N- methylsulfamoyl- amino)-2,6- difluoro-N-(2- methoxypyrazolo [1,5-a]pyrimidin- 6-yl)benzamide | ¹H NMR (400 MHz, CD₃OD) δ 9.43 (s, 1H), 8.43 (s, 1H), 7.69 (m, 1H), 7.13 (m, 1H), 6.08 (s, 1H), 4.01 (s, 3H), 3.21 (m, 2H), 2.82 (s, 3H), 1.11 (t, J = 7.4 Hz, 3H); m/z (APCI-pos) M + 1 = 441.2 | 1 |
| 24 | 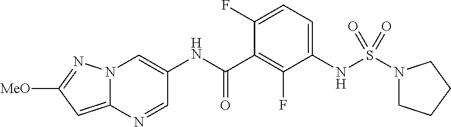 | 2,6-difluoro-N- (2- methoxypyrazolo [1,5-a]pyrimidin- 6-yl)-3- (pyrrolidine-1- sulfonamido) benzamide | ¹H NMR (400 MHz, CD₃OD) δ 9.43 (s, 1H), 8.43 (s, 1H), 7.69 (m, 1H), 7.13 (m, 1H), 6.08 (s, 1H), 4.01 (s, 3H), 3.25 (m, 4H), 1.88 (m, 4H); m/z (APCI- pos) M + 1 = 453.2 | 1 |
| 25 | 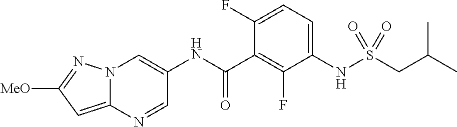 | 2,6-difluoro-N- (2- methoxypyrazolo [1,5-a]pyrimidin- 6-yl)-3-(2- methylpropyl- sulfonamido) benzamide | ¹H NMR (400 MHz, CD₃OD) δ 9.43 (s, 1H), 8.44 (s, 1H), 7.66 (m, 1H), 7.17 (m, 1H), 6.07 (s, 1H), 4.01 (s, 3H), 3.04 (d, J = 7.0 Hz, 2H), 2.28 (m, 1H), 1.10 (d, J = 6.4 Hz, 6H); m/z (APCI-pos) M + 1 = 440.2 | 1 |
| 26 | 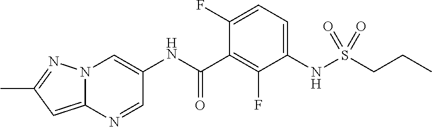 | 2,6-difluoro-N- (2- methylpyrazolo [1,5-a]pyrimidin-6- yl)-3- (propylsulfonamido) benzamide | ¹H NMR (400 MHz, CD₃OD) δ 9.56 (s, 1H), 8.48 (s, 1H), 7.68 (m, 1H), 7.16 (m, 1H), 6.52 (s, 1H), 3.10 (m, 2H), 2.49 (s, 3H), 1.86 (m, 2H), 1.06 (t, J = 7.4 Hz, 3H); m/z (APCI-nega) M − 1 = 408.1 | 1 |
| 27 | 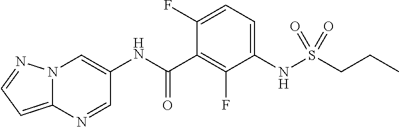 | 2,6-difluoro-3- (propylsulfonamido)- N- (pyrazolo[1,5- a]pyrimidin-6- yl)benzamide | ¹H NMR (400 MHz, (CD₃)₂SO) δ 11.38 (br s, 1H), 9.84 (br s, 1H), 9.59 (s, 1H), 8.59 (s, 1H), 8.21 (s, 1H), 7.60 (m, 1H), 7.31 (m, 1H), 6.77 (s, 1H), 3.13 (m, 2H), 1.77 (m, 2H), 1.00 (t, J = 7.4 Hz, 3H); m/z (APCI- nega) M − 1 = 394.1 | 1 |
| 28 | 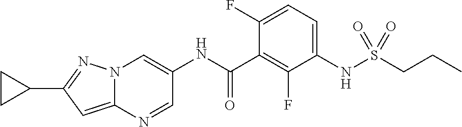 | N-(2- cyclopropylpyrazolo [1,5-a]pyrimidin-6- yl)-2,6-difluoro- 3- (propylsulfonamido) benzamide | ¹H NMR (400 MHz, CD₃OD) δ 9.53 (s, 1H), 8.46 (s, 1H), 7.68 (m, 1H), 7.16 (m, 1H), 6.40 (s, 1H), 3.12 (m, 2H), 2.11 (m, 1H), 1.86 (m, 2H), 1.07 (m, 5H), 0.91 (m, 2H); m/z (APCI- nega) M − 1 = 434.1 | 1 |
| 29 | 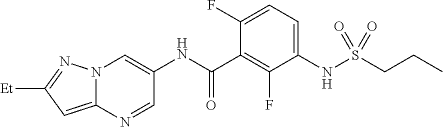 | N-(2- ethylpyrazolo[1,5- a]pyrimidin-6- yl)-2,6-difluoro- 3- (propylsulfonamido) benzamide | ¹H NMR (400 MHz, CD₃OD) δ 9.57 (s, 1H), 8.48 (s, 1H), 7.68 (m, 1H), 7.16 (m, 1H), 6.54 (s, 1H), 3.12 (m, 2H), 2.86 (m, 2H), 1.86 (m, 2H), 1.37 (m, 3H), 1.06 (t, J = 7.4 Hz, 3H); m/z (APCI-nega) M − 1 = 422.0 | 1 |
| 30 | 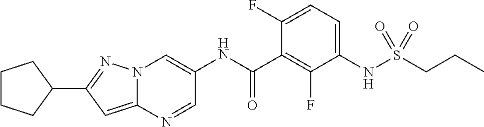 | N-(2- cyclopentylpyrazolo [1,5-a]pyrimidin-6- yl)-2,6-difluoro- 3- (propylsulfonamido) benzamide | ¹H NMR (400 MHz, CD₃OD) δ 9.57 (s, 1H), 8.48 (s, 1H), 7.68 (m, 1H), 7.16 (m, 1H), 6.54 (s, 1H), 3.12 (m, 2H), 2.15 (m, 2H), 1.90-1.74 (m, 9H), 1.06 (t, J = 7.4 Hz, 3H); m/z (APCI- nega) M − 1 = 462.0 | 1 |
| 31 | 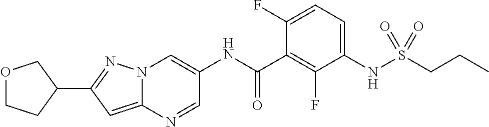 | 2,6-difluoro-3- (propylsulfonamido)- N-(2- (tetrahydrofuran- 3- yl)pyrazolo[1,5- a]pyrimidin-6- yl)benzamide | ¹H NMR (400 MHz, CD₃OD) δ 9.59 (s, 1H), 8.50 (s, 1H), 7.68 (m, 1H), 7.16 (m, 1H), 6.60 (s, 1H), 4.19 (m, 1H), 4.04 (m, 1H), 3.93 (m, 2H), 3.70 (m, 1H), 3.12 (m, 2H), 2.43 (m, 1H), 2.24 (m, 1H), 1.87 (m, 2H), 1.06 (t, J = 7.4 Hz, 3H); m/z (APCI-nega) M − 1 = 464.1 | 1 |
| 32 | 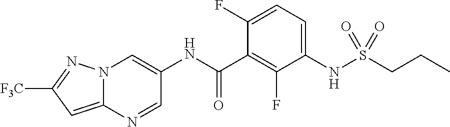 | 2,6-difluoro-3- (propylsulfonamido)- N-(2- (trifluoromethyl) pyrazolo[1,5- a]pyrimidin-6- yl)benzamide | ¹H NMR (400 MHz, CD₃OD) δ 9.77 (s, 1H), 8.67 (s, 1H), 7.68 (m, 1H), 7.16 (m, 1H), 7.06 (s, 1H), 3.12 (m, 2H), 1.87 (m, 2H), 1.06 (t, J = 7.4 Hz, 3H); m/z (APCI-nega) M − 1 = 462.0 | 1 |
| 33 | 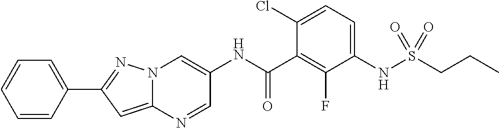 | 6-chloro-2- fluoro-N-(2- phenylpyrazolo [1,5-a]pyrimidin-6- yl)-3- (propylsulfonamido) benzamide | ¹H NMR (400 MHz, (CD₃)₂SO) δ 11.39 (br s, 1H), 10.02 (br s, 1H), 9.59 (s, 1H), 8.59 (s, 1H), 8.05 (m, 2H), 7.61-7.38 (m, 5H), 7.30 (s, 1H), 3.18 (m, 2H), 1.78 (m, 2H), 1.00 (t, J = 7.4 Hz, 3H); m/z (APCI-nega) M − 1 = 486.0, 488.1 | 5 |
| 34 | 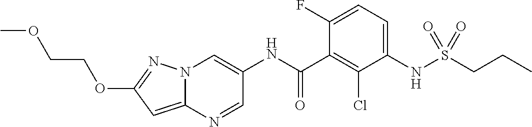 | 2-chloro-6- fluoro-N-(2-(2- methoxyethoxy) pyrazolo[1,5- a]pyrimidin-6- yl)-3- (propylsulfonamido) benzamide | ¹H NMR (400 MHz, CD₃OD) δ 9.44 (s, 1H), 8.43 (s, 1H), 7.72 (m, 1H), 7.29 (m, 1H), 6.09 (s, 1H), 4.43 (m, 2H), 3.79 (m, 2H), 3.43 (s, 3H), 3.12 (m, 2H), 1.88 (m, 2H), 1.06 (t, J = 7.4 Hz, 3H); m/z (APCI-nega) M − 1 = 484.0, 486.0. | 1 |
| 35 | 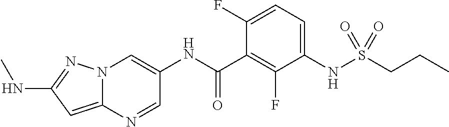 | 2,6-difluoro-N- (2- (methylamino) pyrazolo[1,5- a]pyrimidin-6- yl)-3- (propylsulfonamido) benzamide | ¹H NMR (400 MHz, CD₃OD) δ 9.29 (s, 1H), 8.31 (s, 1H), 7.66 (m, 1H), 7.14 (m, 1H), 5.81 (s, 1H), 3.12 (m, 2H), 2.90 (s, 3H), 1.88 (m, 2H), 1.06 (t, J = 7.4 Hz, 3H); m/z (APCI-pos) M + 1 = 425.1 | 17 |
| 36 | 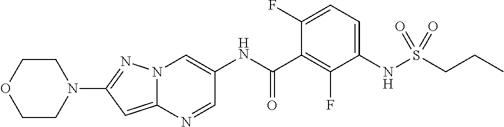 | 2,6-difluoro-N- (2- morpholinopyrazolo [1,5-a] pyrimidin-6- yl)-3- (propylsulfonamido) benzamide | ¹H NMR (400 MHz, (CD₃)₂SO) δ 11.12 (br s, 1H), 9.79 (br s, 1H), 9.25 (s, 1H), 8.37 (s, 1H), 7.54 (m, 1H), 7.26 (m, 1H), 6.14 (s, 1H), 3.71 (m, 4H), 3.30 (m, 4H), 3.12 (m, 2H), 1.75 (m, 2H), 0.97 (t, J = 7.4 Hz, 3H); m/z (APCI-nega) M − 1 = 479.1 | 17 |

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula I: 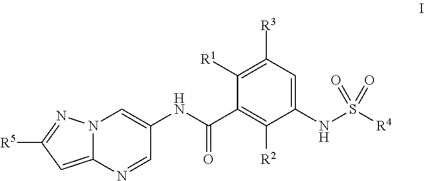
and stereoisomers, tautomers and pharmaceutically acceptable salts thereof, wherein:
R¹ and R² are independently selected from hydrogen, halogen, C₁-C₃ alkyl and C₁-C₃ alkoxy;
R³ is hydrogen, halogen or C₁-C₃ alkyl;
R⁴ is C₃-C₅ cycloalkyl, C₁-C₆ alkyl, C₂-C₆ alkenyl, C₂-C₆ alkynyl, phenyl, a 5-6 membered heteroaryl, or NR^{a}R^{b}, wherein the cycloalkyl, alkyl, alkenyl, alkynyl, phenyl and heteroaryl are optionally substituted with OR^{c}, halogen, phenyl, C₃-C₄ cycloalkyl, or C₁-C₄ alkyl optionally substituted with halogen;
R⁵ is selected from hydrogen, C₁-C₆ alkyl, OR^{d}, NR^{e}R^{f}, SR^{g}, C₃-C₆ cycloalkyl, phenyl, a 4-6 membered heterocyclic and a 5-6 membered heteroaryl, wherein the alkyl, cycloalkyl and heterocyclic are optionally substituted with one to three R^{h} groups, and the phenyl and heteroaryl are optionally substituted with one to three Rⁱ groups;
R^{a} and R^{h} are independently selected from hydrogen and C₁-C₅ alkyl optionally substituted with halogen, or
R^{a} and R^{b} together with the nitrogen to which they are attached form a 4 to 6 membered heterocyclic ring;
R^{c} is hydrogen, phenyl and C₁-C₄ alkyl optionally substituted with oxo;
R^{d} is C₁-C₆ alkyl optionally substituted with OH or OCH₃;
R^{e} and R^{f} are independently selected from hydrogen and C₁-C₆ alkyl;
R^{g} is C₁-C₆ alkyl;
each R^{h} is independently selected from halogen, oxo, C₁-C₆ alkyl, C₁-C₆ alkoxy and a 4-6 membered heterocyclic, wherein the alkyl, alkoxy and heterocyclic are optionally substituted with R^{j};
each Rⁱ is independently selected from halogen, C₁-C₆ alkyl, C₁-C₆ alkoxy and a 4-6 membered heterocyclic, wherein the alkyl, alkoxy and heterocyclic are optionally substituted with R^{k};
R^{j} is selected from halogen, OH, oxo and C₁-C₃ alkyl; and
R^{k} is selected from halogen, OH and C₁-C₃ alkyl.

2. A compound of claim 1, wherein R⁵ is selected from hydrogen, methyl, ethyl, CF₃, —OCH₃, —OCH₂CH₃, —OCH(CH₃)₂, —OCH₂CH₂OH, —OCH₂CH₂OCH₃, —NHCH₃, —NHCH(CH₃)₂, —SCH₃, cyclopropyl, cyclopentyl, phenyl, 4-chlorophenyl, 3-fluorophenyl, 4-fluorophenyl, 4-methylphenyl, 3-(4-methylpiperazin-1-yl)phenyl, tetrahydrofuran-3-yl, pyrrolidin-1-yl, morpholin-4-yl, piperidin-4-yl, 1-methyl-1H-pyrazol-4-yl, 1-(2-hydroxyethyl)-1H-pyrazol-4-yl and pyridin-3-yl.

3. A compound as claimed in claim 1 or 2, wherein R¹, R² and R³ are independently selected from hydrogen, halogen and C₁-C₃ alkyl.

4. A compound as claimed in any one of claims 1 to 3, wherein the residue: 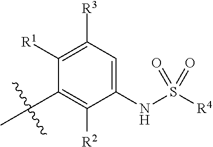
of Formula I, wherein the wavy line represents the point of attachment of the residue in Formula I, is selected from:

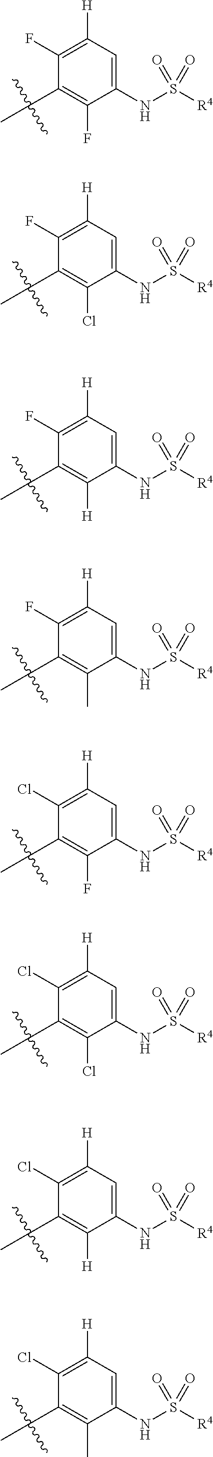

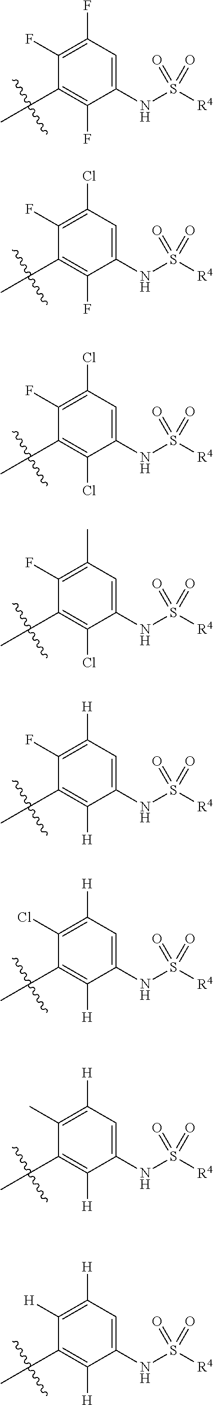

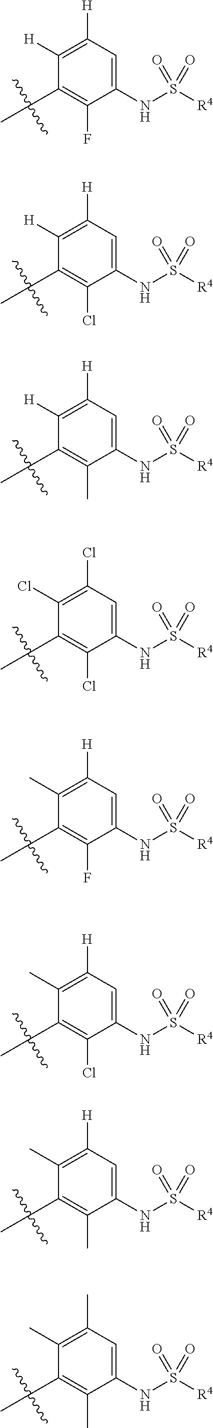

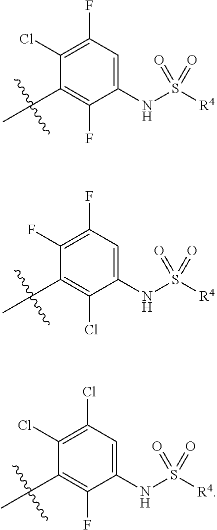

5. A compound as claimed in any one of claims 1 to 4, wherein R¹ is selected from hydrogen, halogen or methyl; R² is Cl; and R³ is hydrogen.

6. A compound as claimed in claim 1 or 2, wherein R¹ and R² are independently selected from halogen, and R³ is hydrogen.

7. A compound as claimed in any one of claims 1 to 6, wherein R⁴ is selected from C₁-C₆ alkyl optionally substituted with halogen, and NR^{a}R^{b}.

8. A compound as claimed in any one of claims 1 to 7, wherein R⁴ is selected from propyl, isobutyl, —CH₂CH₂CH₂F, —N(CH₃)CH₂CH₃ and pyrrolidin-1-yl.

9. A compound of Formula I as defined in claim 1 and named in any one of Examples 1 to 37 herein.

10. A pharmaceutical composition, comprising a compound as claimed in any one of claims 1 to 9, and a pharmaceutically acceptable carrier or excipient.

11. A method of preventing or treating a disease or disorder modulated by b-Raf, comprising administering to a mammal in need of such treatment an effective amount of a compound of any one of claims 1 to 9.

12. A method of preventing or treating cancer, comprising administering to a mammal in need of such treatment an effective amount of a compound of any one of claims 1 to 9, alone or in combination with one or more additional compounds having anti-cancer properties.

13. The method of claim 12, wherein the cancer is a sarcoma.

14. The method of claim 12, wherein the cancer is a carcinoma.

15. The method of claim 14, wherein the carcinoma is squamous cell carcinoma.

16. The method of claim 14, wherein the carcinoma is adenoma or adenocarcinoma.

17. The method of claim 12, wherein the cancer is breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

18. A method of treating a hyperproliferative disease in a mammal comprising administering a therapeutically effective amount of a compound of any one of claims 1 to 9 to the mammal.

19. A compound as claimed in any one of claims 1 to 9 for use in therapy.

20. A compound as claimed in any one of claims 1 to 9 for use in the treatment of a hyperproliferative disease.

21. Use of a compound of any one of claims 1 to 9 in the manufacture of a medicament for the treatment of a hyperproliferative disease.

22. Use of a compound as claimed in any one of claims 1 to 9, in the manufacture of a medicament, for use as a b-Raf inhibitor in the treatment of a patient undergoing cancer therapy.

23. A method of preventing or treating kidney disease, comprising administering to a mammal in need of such treatment an effective amount of a compound of any one of claims 1 to 9, or a stereoisomer, tautomer or pharmaceutically acceptable salt thereof, alone or in combination with one or more additional compounds.

24. The method of claim 23, wherein the kidney disease is polycystic kidney disease.

25. A compound of any one of claims 1 to 9 for use in the treatment of a kidney disease.

26. The compound of claim 25, wherein the kidney disease is polycystic kidney disease.

27. Use of a compound of any one of claims 1 to 9 in the manufacture of a medicament for the treatment of a kidney disease.

28. The use of claim 27, wherein the kidney disease is polycystic kidney disease.

29. A pharmaceutical composition comprising a compound as claimed in any one of claims 1 to 9 for use in the treatment of a hyperproliferative disease.

30. A pharmaceutical composition comprising a compound as claimed in any one of claims 1 to 9 for use in the treatment of cancer.

31. A pharmaceutical composition comprising a compound as claimed in any one of claims 1 to 9 for use in the treatment of kidney disease.

32. The composition of claim 31, wherein the kidney disease is polycystic kidney disease.
